(19) United States
(12) Patent Application Publication
(10) Pub. No.: US 2022/0110979 A1
(43) Pub. Date: Apr. 14, 2022

(54) FIBROBLAST REGENERATIVE CELLS

(71) Applicant: Figene, LLC, Houston, TX (US)

(72) Inventors: Pete O'Heeron, Houston, TX (US); Thomas Ichim, San Diego, CA (US)

(21) Appl. No.: 17/310,007

(22) PCT Filed: Jan. 13, 2020
(86) PCT No.: PCT/US2020/013315

§ 371 Date: Jul. 9, 2021

Related U.S. Application Data
(60) Provisional application No. 62/791,207, filed on Jan. 11, 2019.

(51) Int. Cl. A61K 35/33; A61K 45/06; A61K 35/28; C12N 5/077; C12N 5/0793; C12N 5/071; C12N 5/0789; G01N 33/50

(52) U.S. Cl. CPC A61K 35/33; C12N 2501/14; A61K 35/28; C12N 5/0656; C12N 5/0619; C12N 5/067; C12N 5/069; C12N 5/0647; G01N 33/5005; C12N 2501/115; C12N 2501/119; C12N 2501/41; C12N 2501/13; C12N 2501/12; C12N 2501/165; C12N 2501/155; C12N 2501/125; C12N 2501/26; C12N 2501/2306; C12N 2501/145; A61K 45/06

(57) ABSTRACT

Disclosed are compositions, systems and methods comprising a regenerative fibroblast cell, population or subsets thereof possessing regenerative activity useful for treatment of various degenerative diseases. In one embodiment, the disclosure provides fibroblasts with enhanced proliferative potential based on enrichment for CD105 and/or CD117 markers. In one embodiment, fibroblasts possessing CD105 and/or CD117 markers are further enriched for the property of rhodamine 123 efflux.

This application claims priority to U.S. Provisional Patent Application Ser. No. 62/791,207, filed Jan. 11, 2019, which is incorporated by reference herein in its entirety.

### TECHNICAL FIELD

The present disclosure relates generally to cell biology and medicine. Particularly it concerns regenerative medicine. More particularly, the current disclosure pertains to methods and compositions comprising fibroblasts, fibroblast populations and subpopulations possessing unique regenerative activities.

### BACKGROUND

Regenerative medicine, applied in the form of stem cell therapy, offers the possibility of treating many previously incurable diseases. Numerous types of stem cells exist and there is a need to identify additional stem cells . Generally, stem cells are divided into embryonic and adult types. While embryonic stem cells possess a great ability to proliferate, specific induction of their controlled differentiation has been elusive. The fear of embryonic stem cells causing teratomas has been a major obstacle to their clinical development. Adult stem cells such as bone marrow, cord blood, adipose-derived and amnioticfluid-derived have demonstrated regenerative potential in a variety of diseases and degenerative disorders. However, these cell types are limited by availability, invasiveness of extraction, and in some cases limited proliferative capacity. There is a need for improved cells that overcome these deficiencies and that raise neither issues of oncogenecity nor of karyotypic dysfunction during culture. There is also a need for improved cellular therapies for a variety of medical conditions. The current disclosure overcomes limitations of stem cells by providing a novel regenerative cell, a fibroblast regenerative cell, which possesses superior activity to stem cells. The fibroblast regenerative cell can be utilized for therapeutic purposes.

### BRIEF SUMMARY

Embodiments of the disclosure encompass compositions, methods, and systems comprising fibroblasts possessing regenerative activity that are useful in cellular therapy and in the treatment of various diseases, disorders or conditions. In some embodiments, the fibroblasts have enhanced proliferative potential based on enrichment at least for one or more markers. In specific embodiments, the fibroblasts express CD105 and/or CD117 markers. In some embodiments, fibroblast regenerative cells possessing CD105 and/or CD117 markers are further enriched for the property of rhodamine 123 efflux. In some embodiments, the fibroblast regenerative cells do not express at least one or more of MHC class I, MHC class II, CD45, CD13, CD49c, CD66b, CD73, CD105 or CD90 cell surface proteins.

Embodiments include compositions comprising fibroblast regenerative cells, a population, progeny or subset of fibroblast regenerative cells, a conditioned medium of fibroblast regenerative cells, compositions comprising fibroblast regenerative cells, pharmaceutical formulations comprising fibroblast regenerative cells, kits comprising fibroblast regenerative cells, a cell bank comprising populations of fibroblast regenerative cells, and so forth.

In some aspects, the isolated fibroblast regenerative cells express or are selected because they express CD105 marker and/or CD117 marker. In some embodiments, the cells also express Oct-4, CD-34, KLF-4, Nanog, Sox-2, Rex-1, GDF-3, IL-10, Stella and/or any combination thereof. In some aspects fibroblast regenerative cells also comprise a rhodamine 123 efflux activity. In some aspects, the cells comprise enhanced expression of GDF-11 as compared to a control cell.

In some aspects of the current disclosure, the fibroblast regenerative cells are derived from a tissue having regenerative properties such as, but not limited to, placental tissue, umbilical cord tissue, endometrial cells, Wharton's jelly, bone marrow, adipose tissue, or a mixture thereof. In some aspects, the regenerative fibroblast cells are capable of proliferating and differentiating into at least two of ectoderm, mesoderm, or endoderm. The fibroblast regenerative cells may be autologous or allogeneic with respect to a subject undergoing treatment. In some aspects, the cells are plastic adherent. In further aspects, the cells may be cryopreserved.

Further aspects of the disclosure relate to methods or compositions comprising fibroblasts regenerative cells and exogenous mitochondria. In some aspects, the exogenous mitochondria are isolated and substantially purified from other cellular components. In some aspects, the mitochondria are administered to the fibroblast regenerative cells by lipid fusion, polyethylene glycol-mediated fusion, or by electroporation. In some aspects, the mitochondria are encapsulated or treated with one or more agents to facilitate the incorporation of the mitochondria into the fibroblast cell. The mitochondria can be autologous, syngeneic, allogeneic, or xenogeneic with respect to a subject undergoing treatment. In some aspects, the mitochondria are derived from stem cells, or from cells less differentiated as compared to the fibroblast regenerative cell. In some methods, the mitochondria are administered separately to the subject.

The current disclosure also relates to an isolated fibroblast regenerative cell or population thereof capable of proliferating and differentiating into ectoderm, mesoderm, or endoderm, wherein the isolated fibroblast regenerative cell expresses at least one of Oct-4, Nanog, IL-10, Sox-2, KLF4, c-Myc, Rex-1, GDF-3, LIF receptor, CD105, CD117, CD344 or Stella markers, and does not express at least one of MHC class I, MHC class II, CD45, CD13, CD49c, CD66b, CD73, CD105, or CD90 cell surface proteins.

In certain embodiments, the cells, compositions or formulations of the current disclosure are administered with one or more other compounds that are not fibroblast regenerative cells, a population thereof, progeny thereof and/or a conditioned medium thereof. The one or more other compounds may or may not be in the cells, compositions or formulations encompassed herein. In specific cases, the compounds can be vitamin A, vitamin C, vitamin D, vitamin E, vitamin K, folic acid, choline, vitamin B1, vitamin B2, vitamin B5, vitamin B6, vitamin B12, biotin, nicotinamide, betacarotene, coenzyme Q, selenium, superoxide dismutase, glutathione peroxide, uridine, creatine succinate, pyruvate, dihydroxyacetone), acetyl-L-carnitine, alpha-lipoic acid, cardiolipin, omega fatty acid, lithium carbonate, lithium citrate, calcium, or any combination thereof. In some aspects, the compounds comprise one or more anti-inflammatory agents. In some aspects, the anti-inflammatory agents are one or more of Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; Zomepirac Sodium. In some aspects, the compounds are bioactive compounds including but not limited to growth factors, cytokines, antibodies, antibody fragments, and/or organic molecules, for example of a mass of less than 5000 daltons. The compounds referred to above or elsewhere herein may or may not be administered concurrently with a composition of the current disclosure. Alternatively, the compounds may be administered before and/or after the composition is administered to a subject.

In some embodiments, a composition comprising fibroblasts further comprises one or more types of stem cells, including embryonic stem cells, tissue-specific stem cells, mesenchymal stem cells, or induced pluripotent stem cells, for example. The cells may be provided in the amounts of at least 1×10², 1×10⁶, 1×10⁹, 1×10¹⁰, 1×10¹², 1×10¹⁴ stem cells or any amount in between. The stem cells can be mesenchymal cells, embryonic stem cells or differentiated cells. For example, the stem cells can be myocytes, adipocytes, ectodermal cells, muscle cells, osteoblasts, chondrocytes, endothelial cells, fibroblasts, pancreatic cells, hepatocytes, bile duct cells, bone marrow cells, neural cells, or genitourinary cells, as examples.

Embodiments also include methods of providing cell therapy, methods of treating a subject who would benefit from cell therapy, methods of treating a disease, a disorder or a condition, methods of treating cancer, methods of treating degenerative diseases, methods of transplanting fibroblast regenerative cells, methods for using fibroblast regenerative cells, methods of enhancing the regenerative activity of fibroblast cells, method of generating a population of fibroblast regenerative cells, methods of producing fibroblast regenerative cells, methods for selecting for a fibroblast regenerative cell, methods for identifying a fibroblast regenerative cell, methods for isolating a population of regenerative fibroblast cells, methods of culturing, methods of enriching fibroblast regenerative cells, methods of expanding fibroblast regenerative cells, or a combination thereof. The steps and embodiments discussed in this disclosure are contemplated as part of any of these methods. Moreover, compositions for use in any of these methods are also contemplated.

Particular embodiments include methods of treating at least one condition in a subject by administering to the subject a composition comprising isolated fibroblast regenerative cells, a population thereof, a progeny thereof, and/or a conditioned medium thereof, wherein the fibroblast regenerative cells have an increased regenerative activity compared to a control fibroblast cell.

Methods may comprise or consist of or consist essentially of one or more of the following steps: administering to a subject an effective amount of fibroblast regenerative cells, and administering to the subject one or more additional compounds that enhance or increase or ameliorate the regenerative activity of the fibroblasts of the current disclosure.

In some embodiments, the subject suffers from or is at risk of developing or is suspected of having a condition, disease or disorder. In some embodiments, the subject may have undergone one or more previous treatments for the condition, disease or disorder. In further embodiments, the subject may be resistant to certain other therapies. The condition, disease or disorder may be an immune disorder, a muscular disorder, a hematopoietic disorder, a liver disorder, an angiogenesis disorder, a pancreatic disorder, a cardiac disease, a pulmonary disease, neurological disease, neurological disorder, neurodegenerative disease, muscular disorder, muscular disease, immune disease, inflammatory-mediated disease, inflammatory-mediated disorder, inflammation, ischemia, stroke, ischemic heart disease, liver failure, kidney failure, peripheral artery disease, pulmonary fibrosis, liver fibrosis, pancreatic fibrosis, diabetic limb, fibrosis, scar tissue formation, pathological apoptosis, diabetes, cirrhosis, hepatitis, osteoporosis, a bone fracture, a neurological injury, the need for a prosthesis in a joint, the need for dermal stem cells, the need to increase the proliferation of islet cells, the need to increase proliferation of hepatocytes, the need to increase insulin production, the need for osteocytes, the need to increase osteocyte formation, the need to increase osteocyte function, the need for a neoplastic disease, or the need of cell therapy. In some aspects, the cardiac disease or pulmonary disease is atherosclerosis, myocardial infarction (Heart Attack), cardiac infection, heart failure, ischemic heart failure, high blood pressure (Hypertension), or pulmonary hypertension, idiopathic pulmonary fibrosis, stroke, congenital heart disease (CHD), congestive heart failure, angina, myocarditis, coronary artery disease, cardiomyopathy, dilated cardiomyopathy, hypertrophic cardiomyopathy, endocarditis, diastolic dysfunction, cerebrovascular disease, valve disease, mitral valve prolapse, venous thromboembolism or arrhythmia. In some aspects, the neurological or muscular disease is multiple sclerosis (MS), spinal cord injury, muscular dystrophy (Becker's or Duchenne's), amyotrophic lateral sclerosis (ALS; Lou Gehrig's disease or classical motor neuron disease), autism, progressive bulbar palsy (progressive bulbar atrophy), pseudobulbar palsy, primary lateral sclerosis (PLS), progressive muscular atrophy, spinal muscular atrophy (SMA, including SMA type I—Werdnig-Hoffmann disease, SMA type II, or SMA type III—Kugelberg-Welander disease), Fazio-Londe disease, Kennedy disease (progressive spinobulbar muscular atrophy), congenital SMA with arthrogryposis, and post-polio syndrome (PPS). In some aspects, the immune disorder or the inflammatory-mediated disorder or the immune disease or the inflammatory-mediated disease is thyroiditis, insulitis, multiple sclerosis, iridocyclitis, uveitis, orchitis, Addison's disease, myasthenia gravis, rheumatoid arthritis, lupus erythematosus, immune hyperreactivity, insulin dependent diabetes mellitus, anemia, aplastic anemia, hemolytic anemia, hepatitis, autoimmune hepatitis, skleritis, idiopathic thrombocytopenic purpura, auto immune diseases, diseases of the gastrointestinal tract, Crohn's disease, ulcerative colitis, inflammatory bowel diseases, juvenile arthritis, scleroderma and systemic sclerosis, Sjogren's syndrome, undifferentiated connective tissue syndrome, antiphospholipid syndrome, vasculitis, polyarteritis nodosa, allergic granulomatosis, angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans, polymyalgia rheumatica, es sentiell cryoglobulinemia, Psoriasis vulgaris and psoriatic arthritis, diffuse fasciitis with eosinophilia, diffuse fasciitis without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, inflammatory dermatitis, unwanted immune reactions and inflammation associated with arthritis, rheumatoid arthritis, inflammation associated with hypersensitivity and allergic reactions, systemic lupus erythematosus, collagen diseases, inflammation associated with atherosclerosis, arteriosclerosis, atherosclerotic heart disease, reperfusion injury, vascular inflammatory disorders, respiratory distress syndrome, cardiopulmonary diseases, inflammation associated with peptic ulcer, hepatic fibrosis, liver cirrhosis, hepatic diseases, thyroiditis or other glandular diseases, glomerulonephritis, renal diseases, urologic diseases, otitis, oto-rhino-laryngological diseases, dermatitis, dermal diseases, periodontal diseases, dental diseases, orchitis or epididimo-orchitis, infertility, orchidal trauma, immune related testicular diseases, placental dysfunction, placental insufficiency, habitual abortion, eclampsia, pre-eclampsia, immune-related gynaecological diseases, inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, immune reaction to ocular implants, inflammation reaction against ocular implants, immune-related ophthalmic diseases, inflammatory-related ophthalmic diseases, inflammation associated with autoimmune diseases or conditions or disorders of the central nervous system (CNS) or in any other organ, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, pseudo-tumour cerebri, Down's Syndrome, Huntington's disease, amyotrophic lateral sclerosis, inflammatory components of CNS compression, inflammatory components of CNS compression, inflammatory components of CNS compression, CNS trauma infections of the CNS, inflammatory components of muscular atrophies, inflammatory components of muscular dystrophies, conditions or disorders of the central and peripheral nervous systems, post-traumatic inflammation, septic shock, infectious diseases, inflammatory complications of surgery, inflammatory complications of organ transplant, side effects of surgery, side effects of organ transplants, inflammatory complications of gene therapy, immune complications of gene therapy, inflammatory-related side effects of gene therapy, immune-related side effects of gene therapy, inflammation associated with AIDS, humoral immune response, cellular immune response, monocyte proliferative disease, leukocyte proliferative diseases, leukemia, high amounts of monocytes or lymphocytes, or graft rejection. In some aspects, the graft rejection is after transplantation of natural cells, artificial cells, a natural tissue, an artificial tissue, bone marrow, an organ, a pacemaker, a cornea or a lens. In some aspects, the organ is a liver, a kidney, a heart, or a lung. In some aspects, the neurodegenerative disease is Parkinson's disease, Alzheimer's disease, or Huntington disease. In some aspects, the cardiac or pulmonary disease is artherosclerosis, myocardial infarction, cardiac infection, heart failure, ischemic heart failure, hypertension, pulmonary hypertension, idiopathic pulmonary fibrosis, stroke, congenital heart disease (CHD), congestive heart failure, angina, myocarditis, coronary artery disease, cardiomyopathy, dilated cardiomyopathy, hypertrophic cardiomyopathy, endocarditis, diastolic dysfunction, cerebrovascular disease, valve disease, mitral valve prolapse, venous thromboembolism or arrhythmia.

In some embodiments, the subject is in need of increased hematopoiesis, increased liver activity, increased angiogenesis, controlled inflammation, controlled autoimmunity, and/or has ischemia. The ischemia may be in a cardiac tissue, a pulmonary tissue, a kidney tissue, a brain tissue, muscle tissue, in a limb, or the ischemia may be associated with stroke, ischemic heart disease, liver failure, kidney failure, and peripheral artery disease. In some aspects, the subject has or is at risk of developing a stroke, pulmonary fibrosis, a diabetic limb, an ischemic heart disease, liver failure, kidney failure, peripheral artery disease, diabetes, liver failure, cirrhosis, liver or pancreas fibrosis, or hepatitis, osteoporosis, bone fracture, cardiac disease, pulmonary disease or scar tissue formation. In some aspects, the scar tissue formation or fibrosis is in a pancreatic tissue, a liver tissue, a cardiac tissue, a pulmonary tissue, a limb, liver, pancreas or kidney.

In some aspects, the subject is in need of inhibition, reduction, controlled or reversal of or decreased pathological apoptosis. In some aspects, the subject is in need of improved pancreas function, liver function, osteocyte function, insulin production, pulmonary function, cardiac function or is in need of a prosthesis in a joint, increased number of islet cells, hepatocytes, increased insulin production; and/or an increased number of osteocytes. In some aspects, the subject is in need of dermal stem cells and/or activation of endogenous dermal stem cells. In further aspects, the subject is an animal.

In some embodiments of the methods of the current disclosure, the compositions comprising fibroblast regenerative cells are introduced to the subject parenterally, transdermally, transmucosally, by implantation or by transplantation. For example the compositions are administered intravenously, intramuscularly, intrathecally, intrarterially, intradermally, subcutaneously, intra-pleurally, intra-cranially, intra-ocularly or mucosally.

Further aspects of the disclosure relate to an in vitro method of producing a fibroblast regenerative cell or a population thereof, the method comprising selecting and/or expanding a fibroblast cell that expresses CD117 and/or CD105, wherein the fibroblast cell has increased regenerative activity as compared to a control fibroblast cell, including in at least some cases a fibroblast cell that does not express CD117 and/or CD105. In certain embodiments, the method further comprises a step of selecting the fibroblast regenerative cell for CD34 expression. In other embodiments, the method further comprises a step of selecting the fibroblast regenerative cell for rhodamine 123 efflux activity. In some embodiments, the method further comprises a step of selecting the fibroblast regenerative cell for at least one additional marker selected from the group consisting of Oct-4, CD-34, KLF-4, Nanog, Sox-2, Rex-1, GDF-3, Stella, IL-10, and a combination thereof. In yet some additional embodiments, the method further comprises a step of selecting the fibroblast regenerative cell for enhanced expression of GDF-11 as compared to a control fibroblast cell. In some embodiments of the method, the fibroblast regenerative cell does not express at least one or more of MHC class I, MHC class II, CD45, CD13, CD49c, CD66b, CD73, CD105 or CD90 cell surface proteins. In some embodiments, the method further comprises the step of preparing a therapeutically effective amount of the fibroblast regenerative cell and providing the therapeutically effective amount of the fibroblast regenerative cell, population thereof, progeny thereof, or conditioned medium thereof to a subject in need thereof. In some aspects, the population of fibroblast regenerative cells comprises at least 0.01% to 5% freshly extracted fibroblasts.

Further aspects of the disclosure relate to an in vitro method of forming or regenerating one or more neural cells, comprising the steps of: obtaining a fibroblast regenerative cell or a population thereof by selecting a fibroblast cell that expresses CD117 and/or CD105, wherein the fibroblast cell has increased regenerative activity as compared to a control fibroblast cell; and culturing the fibroblast regenerative cell with a combination of at least two of bFGF, FGF-8, SHH, or BDNF.

In other aspects, the disclosure relates to an in vitro method of forming a hepatocyte cell, comprising the steps of: obtaining a fibroblast regenerative cell or a population thereof by selecting and expanding a fibroblast cell that expresses CD117 and/or CD105, wherein the fibroblast cell has increased regenerative activity as compared to a control fibroblast cell; and culturing said fibroblast regenerative cell with hepatocyte growth factor (HGF) and/or FGF-4.

In other aspects, the disclosure relate to an in vitro method of forming an endothelial cell, comprising the steps of: obtaining a fibroblast regenerative cell or a population thereof by selecting a fibroblast cell that expresses CD117 and/or CD105, wherein the fibroblast cell has increased regenerative activity as compared to a control fibroblast cell; and culturing said fibroblast regenerative cell with vascular endothelial growth factor (VEGF).

The disclosure also relates to an in vitro method of forming a hematopoietic cell, comprising the steps of: obtaining a fibroblast regenerative cell or a population thereof by selecting a fibroblast cell that expresses CD117 and/or CD105, wherein the fibroblast cell has increased regenerative activity as compared to a control fibroblast cell; and culturing said fibroblast regenerative cell with a combination of at least two of bone morphogenic protein-4 (BMP4), VEGF, bFGF, stem cell factor (SCF), Flt3L, hyper IL6, thrombopoietin (TPO) or erythropoietin (EPO).

In certain embodiments of the above in vitro methods, the method further comprises a step of selecting the fibroblast regenerative cell for CD34 expression and/or the method further comprises a step of selecting the fibroblast regenerative cell for rhodamine 123 efflux activity. In some embodiments, the above methods further comprise a step of selecting the fibroblast regenerative cell for at least one additional marker selected from the group consisting of Oct-4, CD-34, KLF-4, Nanog, Sox-2, Rex-1, GDF-3, Stella, IL-10, and a combination thereof. In some embodiments, the methods further comprise a step of selecting the fibroblast regenerative cell for enhanced expression of GDF-11 as compared to a control fibroblast cell. In further embodiments of the above methods, the fibroblast regenerative cell does not express at least one or more of MHC class I, MHC class II, CD45, CD13, CD49c, CD66b, CD73, CD105 or CD90 cell surface proteins.

Further aspects of the current disclosure relate to a composition and/or pharmaceutical formulations comprising an isolated fibroblast cell, a population thereof, a progeny thereof, or a conditioned medium thereof, wherein the fibroblast cell has an increased regenerative activity compared to a control fibroblast cell. In some embodiments, the fibroblast cell expresses CD105 marker and/or CD117 marker. In some aspects, the fibroblast cell further comprises a rhodamine 123 efflux activity and/or further expresses at least one additional marker selected from the group consisting of Oct-4, CD-34, KLF-4, Nanog, Sox-2, Rex-1, GDF-3, Stella, IL-10, and a combination thereof. The fibroblast cell may have enhanced expression of GDF-11 as compared to a control cell. In some embodiments, the population comprises at least 1×10², 1×10⁶, 1×10⁹, 1×10¹⁰, 1×10¹², 1×10¹⁴ cells or any amount in between. In some aspects, the composition is in a NaC1 solution, such as a NaC1 solution that is 0.8%-1% NaCl.

In some aspects of the current disclosure, the regenerative activity of fibroblast regenerative cells is the ability to stimulate angiogenesis. In some aspects, angiogenesis further comprises stimulation of human umbilical vein endothelial cell (HUVEC) proliferation. In some aspects, angiogenesis comprises the production of collateral blood vessels. The collateral blood vessels can be an ischemic cardiac tissue or in an ischemic limb tissue or surrounding an occluded blood vessel.

The current disclosure further provides kits comprising any of the above fibroblast regenerative cells, populations, compositions, progeny, conditioned media or formulations thereof.

In further aspects, the disclosure provides a master cell bank comprising a plurality of packaged population of regenerative fibroblast cells, capable of proliferating and differentiating into ectoderm, mesoderm, or endoderm, wherein the isolated fibroblast regenerative cell expresses at least one of Oct-4, Nanog, Sox-2, KLF4, c-Myc, Rex-1, GDF-3, LIF receptor, CD105, CD117, CD344, IL-10, and Stella, and does not express at least one of MHC class I, MHC class II, CD45, CD13, CD49c, CD66b, CD73, CD105, or CD90 cell surface proteins. In some embodiments, the packaged population includes at least 1×10² or more of the cells of previous aspect.

The current disclosure also relates to embodiments for a method for isolating a population of regenerative fibroblast cells, the method comprising providing a tissue with regenerative activity; and enriching for a population of cells that are about 6-12 micrometers in size, wherein the fibroblast regenerative cells express at least one of Oct-4, Nanog, Sox-2, KLF4, c-Myc, Rex-1, GDF-3, LIF receptor, CD105, CD117, CD344, IL-10, and Stella, and does not express at least one of MHC class I, MHC class II, CD45, CD13, CD49c, CD66b, CD73, CD105, or CD90 cell surface proteins. In some embodiments, the method optionally includes the step of depleting cells from the population expressing stem cell surface markers or MHC proteins, thereby isolating a population of stem cells. In some aspects, the cells to be depleted express MHC class I, CD66b, glycophorin a, or glycophorin b. In some aspects, the method optionally includes transfecting the cells with a polynucleotide vector containing a stem cell-specific promoter operably linked to a reporter or selection gene. In some aspects, the cell-specific promoter is an Oct-4, Nanog, Sox-9, GDF3, Rex-1, or Sox-2 promoter. In some embodiments, the method further includes the step of enriching the population for the regenerative fibroblast cells using expression of a reporter or selection gene. In some embodiments, the method further includes the step of enriching the population of the regenerative fibroblast cells by flow cytometry. In some embodiments, the method further includes the steps of contacting the cells with a detectable compound that enters the cells, the compound being selectively detectable in proliferating and non-proliferating cells; and enriching the population of cells for the proliferating cells. In some aspects, the detectable compound is carboxyfluorescein diacetate, succinimidyl ester, or Aldefluor. In some embodiments, method further includes culturing the cells under conditions that form tissue aggregate bodies. In some embodiments, the method further includes culturing the population of fibroblast regenerative cells under conditions that support proliferation of the cells.

In some embodiments, the method further includes separating cell types such as granulocytes, T-cells, B-cells, NK-cell, red blood cells, or any combination thereof, from the fibroblast regenerative cells. In some aspects, separating the cell types is done by cell depletion.

Further embodiments of the current disclosure relate to a method of identifying a fibroblast regenerative cell, the method comprises the steps of introducing into a cell a vector comprising a fibroblast cell-specific promoter coupled to at least one selectable marker gene; expressing the selectable marker gene from the cell specific promoter in the cell; and detecting expression of the marker gene in the cell, thereby identifying the fibroblast regenerative cell, wherein said fibroblast regenerative cell does not express at least one or more of MHC class I, MHC class II, CD44, CD45, CD13, CD34, CD49c, CD66b, CD73, CD105, and CD90 cell surface proteins; and said fibroblast regenerative cell expresses at least one or more of Oct-4, Nanog, Sox-2, Rex-1, GDF-3, Stella, FoxD3, IL-10, or Polycomb embryonic transcription factors, and wherein said fibroblast regenerative cell is capable of differentiating into mesoderm, ectoderm, and/or endoderm. In some embodiments, the fibroblast cell does not express CD13, CD44, CD90, or a combination thereof.

In some embodiments, the fibroblast cell-specific promoter is an Oct-4 promoter, a Nanog promoter, a Sox-2 promoter, a Rex-1 promoter, a GDF-3 promoter, a Stella promoter, a FoxD3 promoter, a Polycomb Repressor Complex 2 promoter, an IL-10 promoter, or aCTCF promoter. In some embodiments, the fibroblast cell-specific promoter is flanked by loxP sites.

In some embodiments, the method further comprises the step of isolating the fibroblast regenerative cell. In some aspects the fibroblast regenerative cell is derived from the bodily fluid and/or from the tissue of a mammal. In some embodiments, the bodily fluid is synovial fluid or blood. In some embodiments, the mammal is a human.

In some embodiments, the vector is a retroviral vector. In some embodiments, the selectable marker gene encodes a fluorescent protein, such as but limited to Green Fluorescent Protein (GFP). In some embodiments, the vector comprises two selectable marker genes, the two selectable marker genes comprise a fluorescent protein, a protein sensitive to drug selection, a cell surface protein or any combination thereof.

Further aspects of the disclosure relate to a method of generating a regenerative fibroblast cell comprising the steps of introducing into a population of fibroblasts a vector comprising a promoter, such as a regenerative cell-specific promoter, coupled to at least one selectable marker gene, wherein said regenerative cell does not express MHC class I, MHC class II, CD44, CD45, CD13, CD34, CD49c, CD73, CD105 and CD90 cell surface proteins; expressing the selectable marker gene from the regenerative-cell specific promoter in said fibroblast population; and detecting expression of the marker gene in the regenerative fibroblast cell.

In some embodiments, the methods further comprise a step of transfecting the regenerative fibroblast cells with OCT-4 transcription factor, thereby enhancing the regenerative activity of the fibroblast cells.

In some embodiments, the methods further comprise a step of fusing the regenerative fibroblast cells with cells having a pluripotent ability thereby generating fibroblasts with enhanced regenerative activity.

In some embodiments, the methods further comprise the steps of selecting fibroblast cells expressing CD105 and/or CD 117; and transfecting the fibroblast cells expressing CD105 and/or CD 117 with permeant NANOG gene. In some aspects, the fibroblast regenerative cell further comprises a rhodamine 123 efflux activity. In further aspects, the fibroblast regenerative cell has enhanced expression of GDF-11 as compared to a control

Use of the one or more compositions may be employed based on methods described herein. Use of one or more compositions may be employed in the preparation of medicaments for treatments according to the methods described herein. Other embodiments are discussed throughout this application. Any embodiment discussed with respect to one aspect of the disclosure applies to other aspects of the disclosure as well and vice versa.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows enhanced expression of OCT-4 in CD105 purified fibroblasts. Control is the left bar, CD105-negative is the middle bar, and CD105-positive is the right bar.

FIG. 2 demonstrates enhanced expression of NANOG in CD105 purified fibroblasts. Control is the left bar, CD105-negative is the middle bar, and CD105-positive is the right bar.

FIG. 3 shows enhanced expression of KLF-4 in CD105 purified fibroblasts. Control is the left bar, CD105-negative is the middle bar, and CD105-positive is the right bar.

FIG. 4 demonstrates enhanced expression of Immune modulatory cytokine IL-10 by CD105 selected fibroblasts. Control is the left bar, CD105-negative is the middle bar, and CD105-positive is the right bar.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### I. Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the term “cell surface protein” refers to a protein that is expressed on the surface of a cell.

As used herein, the term “expansion” refers to the propagation of a cell or cells without terminal differentiation.

As used herein, the term “differentiation” refers to the developmental process of lineage commitment. A “lineage” refers to a pathway of cellular development, in which precursor or “progenitor” cells undergo progressive physiological changes to become a specified cell type having a characteristic function (e.g., nerve cell, muscle cell, or endothelial cell). Differentiation occurs in stages, whereby cells gradually become more specified until they reach full maturity, which is also referred to as “terminal differentiation.” A “terminally differentiated cell” is a cell that has committed to a specific lineage, and has reached the end stage of differentiation (i.e., a cell that has fully matured). By “committed” or “differentiated” is meant a cell that expresses one or more markers or other characteristic of a cell of a particular lineage.

As used herein, the term “isolated” refers to a stem cell or population of daughter stem cells in a non-naturally occurring state outside of the body (e.g., isolated from the body or a biological sample from the body). The biological sample can include synovial fluid, blood (e.g., peripheral blood), or tissue.

As used herein, the term “purified” as in a “purified cell” refers to a cell that has been separated from the body of a subject but remains in the presence of other cell types also obtained from the body of the subject. By “substantially purified” is meant that the desired cells are enriched by at least 20%, more preferably by at least 50%, even more preferably by at least 75%, and most preferably by at least 90% or even 95%.

By a “population of cells” is meant a collection of at least ten cells. Preferably, the population consists of at least twenty cells, more preferably at least one hundred cells, and most preferably at least one thousand, or even one million cells. Because the stem cells of the present invention exhibit a capacity for self-renewal, they can be expanded in culture to produce populations of even billions of cells.

“Germ layers” are the three primary layers formed as a result of gastrulation in early stage embryos, consisting of endoderm, mesoderm, and ectoderm. Embryonic germ layers are the source from which all tissues and organs derive. The endoderm is the source of, for example, pharynx, esophagus, stomach, intestine and associated glands (e.g., salivary glands), liver, epithelial linings of respiratory passages and gastrointestinal tract, pancreas and lungs. The mesoderm is the source of, for example, smooth and striated muscle, connective tissue, vessels, the cardiovascular system, blood cells, bone marrow, skeleton, reproductive organs and excretory organs. Ectoderm is the source of, for example, epidermis (epidermal layer of the skin), sensory organs, the entire nervous system, including brain, spinal cord, and all the outlying components of the nervous system.

The term “multipotent,” with respect to stem cells of the invention, refers to the ability of the stem cells to give rise to cells of all three primitive germ layers (endoderm, mesoderm, and ectoderm) upon differentiation.

The term “allogeneic,” as used herein, refers to cells of the same species that differ genetically from cells of a host.

The term “autologous,” as used herein, refers to cells derived from the same subject. The term “engraft” as used herein refers to the process of stem cell incorporation into a tissue of interest in vivo through contact with existing cells of the tissue.

By “does not detectably express” means that expression of a protein or gene cannot be detected by standard methods. In the case of cell surface markers, expression can be measured by, e.g., flow cytometry, using a cut-off values as obtained from negative controls (i.e., cells known to lack the antigen of interest) or by isotype controls (i.e., measuring nonspecific binding of the antibody to the cell). Thus, a cell that “does not detectably express” a marker appears similar to the negative control for that marker. For gene expression, a gene “does not detectably express” if the presence of its mRNA cannot be visually detected on a standard agarose gel following standard PCR protocols.

Conversely, a cell “expresses” the protein or gene if it can be detected, including by the same method.

The term “culture expanded population” means a population of cells whose numbers have been increased by cell division in vitro. This term may apply to stem cell populations and non-stem cell populations alike.

The term “passaging” refers to the process of transferring a portion of cells from one culture vessel into a new culture vessel.

The term “cryopreserve” refers to preserving cells for long term storage in a cryoprotectant at low temperature.

The term “master cell bank” refers to a collection of cryopreserved cells. Such a cell bank may comprise stem cells, non-stem cells, and/or a mixture of stem cells and non-stem cells.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “activated fibroblasts” refers to fibroblasts treated with one or more stimuli capable of inducing one or more alterations in the cell: metabolic, immunological, growth factor-secreting, surface marker expression, and/or production of microvesicles.

As used herein, the term “activated immune cells” refers to immune cells treated with one or more stimuli capable of inducing one or more alterations in the cell: metabolic, immunological, growth factor secreting, surface marker expression, and production of microvesicles.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, oral ingestion, transdermal patch, topical, inhalation, suppository etc.

As used herein, “allogeneic” refers to tissues or cells from another body that in a natural setting are immunologically incompatible or capable of being immunologically incompatible, although from one or more individuals of the same species.

As used herein, the term “allotransplantation” refers to the transplantation of organs, tissues, and/or cells from a donor to a recipient, where the donor and recipient are different individuals, but of the same species. Tissue transplanted by such procedures is referred to as an allograft or allotransplant.

As used herein, the terms “allostimulatory” and “alloreactive” refer to stimulation and reaction of the immune system in response to an allologous antigens, or “alloantigens” or cells expressing a dissimilar HLA haplotype.

As used herein, the term “angiogenesis” refers to a physiological process involving the growth of new blood vessels from pre-existing vessels and includes initiating angiogenesis, the formation of new blood vessel by initiating from existing ones, and splitting angiogenesis (intussusception: the formation of new blood vessel by splitting off existing ones).

As used herein, the term “autoimmunity” refers to the system of immune responses of an organism against its own healthy cells and tissues.

As used herein, “autologous” refers to tissues or cells that are derived or transferred from the same individual's body (i.e., autologous blood donation; an autologous bone marrow transplant).

As used herein, the term “autotransplantation” refers to the transplantation of organs, tissues, and/or cells from one part of the body in an individual to another part in the same individual, i.e., the donor and recipient are the same individual. Tissue transplanted by such “autologous” procedures is referred to as an autograft or autotransplant.

The term “biologically active” or “bioactive compound” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells that lack protein activity with an expression vector that expresses the protein, a derivative thereof, or a portion thereof. In other cases, a fragment of a gene product (such as a protein) may be considered biologically active (or it may be referred to as functionally active) if it retains the activity of the full-length gene product, although it may be at a reduced but detectable level of the activity of the full-length gene product.

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO2. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

As used herein, the term “collateralization” refers to the growth of a blood vessel or several blood vessels that serve the same end organ or vascular bed as another blood vessel that cannot adequately supply that end organ or vascular bed sufficiently

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “conditioned medium of fibroblast regenerative cells” refers to a liquid media which has been in contact with cells, wherein said cells produce factors which enter the media, thus bestowing upon the media therapeutic activity.

The term “drug”, “agent” or “compound” as used herein, refers to any pharmacologically active substance capable of being administered that achieves a desired effect. Drugs or compounds can be synthetic or naturally occurring, non-peptide, proteins or peptides, oligonucleotides, or nucleotides (DNA and/or RNA), polysaccharides or sugars.

“Growth factor” can be a naturally occurring, endogenous or exogenous protein, or recombinant protein, capable of stimulating cellular proliferation and/or cellular differentiation and/or cellular migration.

The term “subject” or “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

As used herein, the term “ischemia” or “ischemic condition” refers to an inadequate blood supply to an organ or part of the body. Such conditions may comprise cardiac ischemia, ischemic colitis, mesenteric ischemia, ischemic stroke, brain ischemia, renal ischemia, and limb ischemia.

“Mesenchymal stem cell” or “MSC” refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage, for example. In specific embodiments, mesenchymal stem cells do not express substantial (such as more than 50% compared to baseline) levels of HLA-DR, CD117, CD45, or a combination thereof. As used herein, “mesenchymal stromal cell” (which may also be referred to as “mesenchymal stem cell”) or “MSC” can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. As used herein, “mesenchymal stromal cell” or “MSC” includes cells that are CD34 positive upon initial isolation from tissue but are similar to cells described about phenotypically and functionally. As used herein, “MSC” includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. As used herein, “mesenchymal stromal cell” or “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, Stempeuce;CLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. As a non-limiting example, an effective amount is an amount sufficient to promote formation of a blood supply sufficient to support the transplanted tissue. As another non-limiting example, an effective amount is an amount sufficient to promote formation of new blood vessels and associated vasculature (angiogenesis) and/or an amount sufficient to promote repair or remodeling of existing blood vessels and associated vasculature. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, “treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

The term “selecting”, or “selecting for expression” means isolated specific cell(s) from a heterogenous population of other cells, such as by utilizing one or more molecules specific to the cells.

### II. Cell Expansion

Any cells encompassed herein may be expanded in any suitable manner, including using various in vitro systems. In one embodiment, the means of in vitro expansion includes one or more bioreactors. Bioreactors for use in the disclosure are designed to provide a culture process that can deliver medium and oxygenation at controlled concentrations and rates that mimic nutrient concentrations and rates in vivo. Bioreactors have been available commercially for many years and employ a variety of types of culture technologies. Of the different bioreactors used for mammalian cell culture, most have been designed to allow for the production of high density cultures of a single cell type and as such find use in the present invention. For use of the invention, culture techniques and bioreactors used for expansion of mesenchymal stem cells may be applied for use in regenerative fibroblasts, some means are described in references 1-8.

In one embodiment, typical application of these high density systems is to produce as the end-product, a conditioned medium produced by the cells. This is the case, for example, with hybridoma production of monoclonal antibodies and with packaging cell lines for viral vector production. However, these applications differ from applications where the therapeutic end-product is the harvested cells themselves, as in some embodiments of the present disclosure. Once operational, bioreactors provide automatically regulated medium flow, oxygen delivery, and temperature and pH controls, and they generally allow for production of large numbers of cells. Bioreactors thus provide economies of labor and minimization of the potential for mid-process contamination, and the most sophisticated bioreactors allow for set-up, growth, selection and harvest procedures that involve minimal manual labor requirements and open processing steps [9]. Such bioreactors optimally are designed for use with a homogeneous cell mixture or aggregated cell populations as contemplated by the present invention. Suitable bioreactors for use in the present invention include but are not limited to those described in U.S. Pat. Nos. 5,763,194, 5,985,653 and 6,238,908, 5,512,480, 5,459,069, 5,763,266, 5,888,807 and 5,688,687, herein incorporated by reference. With any large volume, several fundamental parameters require control. Cultures must be provided with medium that allows for cell viability maintenance, proliferation and differentiation as well as final cell culture preservation. Typically, the various media are delivered to the cells by a pumping mechanism in the bioreactor, feeding and exchanging the medium on a regular basis [10]. Quantification of metabolites may be performed in order to allow for optimum culture conditions [11]. The exchange process allows for by-products to be removed from the culture.

Growing cells or tissue also requires a source of oxygen. Different cell types can have different oxygen requirements. Accordingly, a flexible and adjustable means for providing oxygen to the cells is a desired component.

Depending on the particular culture, even distribution of the cell population and medium supply in the culture chamber can be an important process control [12, 13]. Such control is often achieved by use of a suspension culture design, which can be effective where cell-to-cell interactions are not important. Examples of suspension culture systems include various tank reactor designs and gas-permeable plastic bags. For cells that do not require assembly into a three-dimensional structure or require proximity to a stromal or feeder layer (such as most blood cell precursors or mature blood cells) such suspension designs may be used. Efficient collection of the cells at the completion of the culture process is an important feature of an effective cell culture system. One approach for production of cells as a product is to culture the cells in a defined space, without physical barriers to recovery, such that simple elution of the cell product results in a manageable, concentrated volume of cells amenable to final washing in a commercial, closed system cell washer designed for the purpose [14]. In specific embodiments, the system would allow for addition of a pharmaceutically acceptable carrier, with or without preservative, or a cell storage compound, as well as provide efficient harvesting into appropriate sterile packaging. In at least some cases, the harvest and packaging process may be completed without breaking the sterile barrier of the fluid path of the culture chamber [15, 16]. In some embodiments expansion of cells is performed using microcarrier beads [17-23]. In some embodiments of the invention cells are grown in media lacking xenogeneic products, referred to as “xeno-free” media. In some embodiments xeno-free media is generated using platelet lysate or other defined conditions [24-26].

In some embodiments of the disclosure, cells are grown under hypoxic conditions in order to allow for enhanced expansion and augmented regenerative activity [27].

### III. Mitochondria

In some embodiments of the disclosure, fibroblast regenerative cells, compositions or pharmaceutical formulations are augmented by the introduction or co-administration of exogenous mitochondria. Isolated and substantially pure mitochondria can be administered to fibroblast regenerative cells through various means such as lipid fusion, polyethylene glycol mediated fusion or electroporation. In other embodiments of the methods of the disclosure, mitochondria may be encapsulated, or treated with agents to facilitate incorporation of said mitochondria into the methods further include administering to the subject one or more agents selected from vitamin A, vitamin C, vitamin D, vitamin E, vitamin K, folic acid, choline, vitamin B1, vitamin B2, vitamin B5, vitamin B6, vitamin B12, biotin, nicotinamide, betacarotene, coenzyme Q, selenium, superoxide dismutase, glutathione peroxide, uridine, creatine succinate, pyruvate, dihydroxyacetone), acetyl-L-carnitine, alpha-lipoic acid, cardiolipin, omega fatty acid, lithium carbonate, lithium citrate, calcium, and mixtures thereof, such as the cocktails described herein. In any of the above aspects, the mitochondria can be syngeneic mitochondria, allogeneic mitochondria, or xenogeneic mitochondria. The mitochondria for use in the methods, kits, and compositions of the invention can be obtained from any source described herein. Means of achieving mitochondrial transfer are known in the art and described in references 28-38. In some embodiments of the disclosure, fibroblasts are recipients of mitochondria derived from stem cells, or cells more undifferentiated as compared to said fibroblasts as shown in references 39-50. All said references are herein incorporated in their entirety by reference.

In certain embodiments, methods include the steps of (i) separating mitochondria from other constituents of a cell to produce isolated and substantially pure mitochondria; and (ii) administering the isolated and substantially pure mitochondria into the subject, such as with the fibroblasts. In certain embodiments, the cells are progenitor cells or any other cell type described herein.

### IV. Cellular Transplantation Therapy

Any medical condition, disease, or disorder may be treated by methods of the disclosure for which fibroblast regenerative cells are therapeutic or prophylactic.

As one example, autoimmune diseases are characterized by an excessive reaction of the immune system against endogenous tissue. The immune system erroneously recognizes endogenous tissue as foreign bodies to be combated. This results in severe inflammatory reactions, which lead to damage to organs affected by them. An important part in distinguishing between endogenous and exogenous structures is played by T lymphocytes or T cells, which are “trained” in the thymus to dock only onto endogenous cell surface molecules, the so-called MHC molecules, and thus to tolerate endogenous structures.

In autoimmune diseases, a group of T cells behaves abnormally. In addition to the still functioning defense from exogenous molecules and organisms, they now also attack endogenous structure. Organs or tissues are perceived as exogenous. There can be various consequences: if vital structures are affected, an autoimmune disease will take a fatal course. The immune system directs its defense against these structures, cellular and also humoral defense reactions are set in motion, and autoantibodies are formed, as a result of which the organs affected in the course of time cease to function. Most commonly, the immune system is weakened and the body becomes susceptible to all kinds of diseases. Under some circumstances, recognition of the exogenous is also disrupted, and as a result the spreading of degenerated cancer cells (for example) can no longer be effectively prevented, and those affected are more susceptible to infectious diseases. In the course of the disease, cells of the immune system destroy the endogenous structures, while the body's repair mechanisms attempt as far as possible to regenerate the damaged organ parts. As a rule, without treatment this erroneous attack of the defensive system continues throughout life or until the complete destruction of the target structure.

Autoimmune diseases are treated according to the organ affected. In this, the basic principle of the causal therapy is to suppress the activity of the immune system by administration of immunosuppressants, e.g., cortisone. These substances are characterized by multiple systemic side-effects and interactions, owing to which attempts have been made to develop new drugs which specifically influence the mechanisms involved in the disease event.

In one embodiment of the disclosure, fibroblasts regenerative cells are administered to an individual for treatment of an autoimmunity or inflammatory disorder. In some embodiments of the disclosure, fibroblast cells are cultured ex vivo and subjected to conditions that reduce their immunogenicity, and then the fibroblasts are utilized to stimulate anti-inflammatory and/or immunomodulatory properties. Additional embodiments are directed to methods of administration of the cells to an individual in need thereof for the purpose of treating an autoimmune and/or inflammatory condition. .

Embodiments of the disclosure provide means of utilizing fibroblasts as allogeneic therapeutic cells through modification of culture conditions in order to decrease immunogenicity of the fibroblasts. In one embodiment of the disclosure, fibroblasts are extracted from sources with lower immunogenicity (e.g. placental fibroblasts, etc.). In another embodiment, fibroblasts are subjected to interferon gamma (IFN-gamma), such as upon culture ex vivo, which without being restricted to mechanism, encompasses reducing immunogenicity. The reduction in immunogenicity may be exemplified by inhibiting the ability of the fibroblasts to evoke alloreactive T cell responses, in specific embodiments. In specific embodiments of the disclosure, these modified fibroblast cells are universal donor fibroblasts.

Embodiments of the present disclosure are directed to systems and methods for the use of fibroblast cells, either autologous or allogeneic, for treatment of inflammatory and/or autoimmune conditions. Methods and compositions of the disclosure encompass certain manipulated cells for the treatment of inflammatory and/or autoimmune conditions. In particular, the cells include at least fibroblasts of any kind. Means of manipulation of fibroblasts are disclosed, as well as fibroblasts of different tissue origins, which actively inhibit inflammatory and/or autoimmune processes. In one embodiment of the disclosure, fibroblasts are utilized as a cellular therapy for prevention and/or treatment of autoimmune conditions. In a specific embodiment, fibroblasts are treated with one or more particular agents and/or conditions to be able to directly or indirectly treat inflammatory and/or autoimmune processes. The route of administration, dosage and frequency is determined as a function of the disease process, as well as stage of the disease, and can be optimized per routine practices in medicine.

In one embodiment, allogeneic fibroblasts are administered to an individual in a non-manipulated manner (for example, without prior exposure to one or more particular agents, such as interferon gamma) but selected from sources naturally characterized by immune modulatory activity, such as placental fibroblasts or adipose tissue-associated fibroblasts, for example. In other embodiments of the disclosure, any fibroblasts are cultured under conditions capable of inducing retro-differentiation so as to endow an immature phenotype for the fibroblasts, wherein the immature phenotype correlates with enhanced anti-inflammatory and/or immune modulatory potential. For example, fibroblasts may be cultured in the presence of one or more histone deacetylase inhibitors, such as valproic acid (Moon et al., 2008; Huang et al., 2011). In addition to HDAC inhibitors, other means of inducing dedifferentiation of the fibroblasts may also be utilized in the context of the current disclosure, such as 8-Br-cAMP (Wang et al., 2011); M-CSF treatment (Li et al., 2016); exposure to reveresine (Li et al., 2016); and/or exposure to stem cell extracts (Xiong et al., 2014). Characterization of fibroblast dedifferentiation can be performed by assessment of extracellular markers, such as , such as CXCR4, VEGFR-2, CD34, and/or CD133, as well as intracellular markers such as SOX-2, NANOG, and/or OCT-4.

In one embodiment of the disclosure, fibroblasts are cultured ex vivo using means known in the art for preserving viability and proliferative ability of fibroblasts. The disclosure provides for the modification of known culture techniques to decrease recognition of fibroblasts by the recipient immune system. In one embodiment fibroblasts are cultured in conditions that lack xenogeneic components, such as fetal calf serum. Xenogeneic components are known to trigger immunological reactions, including elicitation of antibody and T cell reactions (Selvaggi et al., 1997; Mackensen et al., 2000; Kadri et al., 2007; Forni et al., 1976; Lauer et al., 1983). In many individuals, natural antibodies of the IgM isotype exist to fetal calf serum associated components (Irie et al., 1974), causing rejection, inflammation or anaphylaxis subsequent to administration of cells grown in the presence of fetal calf serum (Macy et al., 1989). In specific embodiments, the disclosure encompasses the substitution of fetal calf serum with human platelet rich plasma, platelet lysate, umbilical cord blood serum, autologous serum, and/or defined cytokine mixes as an additional feature to reduce the immunogenicity of fibroblasts. Means of culturing tissues in xenogeneic-free medium are known in the art for other cell types and are incorporated by reference (Riordan et al., 2015).

Embodiments of the disclosure provide methods and compositions comprising fibroblasts. Fibroblasts may be derived from various tissues or organs, such as skin, heart, blood vessels, bone marrow, skeletal muscle, liver, pancreas, brain, and/or foreskin, which can be obtained by biopsy (where appropriate) or upon autopsy. In some aspects, the cells comprise fibroblasts, which can be from a fetal, neonatal, adult origin, or a combination thereof, for example.

Various quality control means are known in the art for practitioners of the disclosure to perform clinical administration of the cells. Example criteria for qualification of the cells includes marker identification using means such as flow cytometry, viability, and/or endotoxin content, as well as assessment for microbial and mycoplasma contamination.

In one embodiment of the disclosure, universal donor fibroblasts are administered to an individual for treatment of an autoimmunity or inflammatory disorder. In some embodiments of the disclosure, cells are cultured ex vivo and subjected to conditions that reduce immunogenicity and stimulate anti-inflammatory and/or immunomodulatory properties. Additional embodiments are directed to methods of administration of the cells to an individual in need thereof for the purpose of treating an autoimmune and/or inflammatory condition.

### V. Various Embodiments

In accordance with the current disclosure, methods of providing a cell therapy and methods of treating a subject that would benefit from a cell therapy are provided. In one embodiment, a method includes administering fibroblast regenerative cells, a population or plurality or culture of fibroblast regenerative cells, progeny of fibroblast regenerative cells or conditioned medium of fibroblast regenerative cells to the subject in an amount sufficient to provide a benefit to the subject. In particular non-limiting aspects, a subject is in need of increased, stimulated, induced, promoted, augmented or enhanced hematopoiesis. In additional non-limiting aspects, a subject is in need of increased, stimulated, induced, promoted, augmented or enhanced liver function or activity; in need of reduced, decreased, inhibited, blocked, prevented, controlled or limited inflammation or autoimmunity; or in need of increased, stimulated, induced, promoted, augmented or enhanced angiogenesis. Thus, methods of the disclosure include administering fibroblast regenerative cells, a population or plurality or culture of fibroblast regenerative cells, progeny of fibroblast regenerative cells or conditioned medium of fibroblast regenerative cells to increase, stimulate, induce, promote, augment or enhance hematopoiesis (in a deficient subject); to increase, stimulate, induce, promote, augment or enhance liver function or activity; to reduce, decrease, inhibit, block, prevent, control or limit inflammation (e.g., to a subject in need of inhibition of inflammation); and to increase, stimulate, induce, promote, augment or enhance angiogenesis. For example, fibroblast regenerative cells can be administered (e.g., intravenously) to a subject with ischemia, so as to induce angiogenesis (e.g., by homing to ischemic tissue in the subject). Numerous diseases have been associated with ischemia, including stroke, ischemic heart disease, liver failure, kidney failure, and peripheral artery disease. Further, methods of the disclosure include administering fibroblast regenerative cells, a population or plurality or culture of fibroblast regenerative cells, progeny of fibroblast regenerative cells or conditioned medium of fibroblast regenerative cells to treat a subject having or at risk of having ischemia in a tissue or organ (e.g., cardiac or pulmonary tissue, limb, or kidney); to treat a subject having or at risk of having a stroke, pulmonary fibrosis, or diabetic limb; to treat a subject in need of inhibition of fibrosis or scar tissue formation; to treat a subject having or at risk of having fibrosis or scar tissue formation in a tissue or organ (e.g., cardiac or pulmonary, limb, liver, pancreas, or kidney); to treat a subject in need of inhibition, reduction, decreased, controlled or reversal of pathological apoptosis; to treat a subject in need of increasing or improving a pancreas or liver function; to increase numbers or proliferation of islet cells, increase numbers or proliferation of hepatocytes, or increase insulin production; to treat a subject having or at risk of having diabetes, liver failure, cirrhosis, liver or pancreas fibrosis, or hepatitis; to treat a subject in need of osteocytes or an osteocyte function (e.g., to increase, stimulate, induce, promote, augment or enhance osteocyte numbers, osteocyte formation or osteocyte function); to treat a subject having or at risk of having osteoporosis, a bone fracture or break, or is in need of a prosthesis in a joint; and to treat a subject in need of dermal stem cells, or activation or stimulation of endogenous dermal stem cells. Moreover, methods of the invention include administering fibroblast regenerative cells, a population or plurality or culture of fibroblast regenerative cells, progeny of fibroblast regenerative cells or conditioned medium of fibroblast regenerative cells to treat a subject in need of increased or improved pulmonary or cardiac function, for example, a subject that has or is at risk of having a cardiac or pulmonary disease. Non-limiting examples of cardiac and pulmonary diseases include artherosclerosis, myocardial infarction (Heart Attack), cardiac infection, heart failure, ischemic heart failure, high blood pressure (Hypertension), or pulmonary hypertension, idiopathic pulmonary fibrosis, stroke, congenital heart disease (CHD), congestive heart failure, angina, myocarditis, coronary artery disease, cardiomyopathy, dilated cardiomyopathy, hypertrophic cardiomyopathy, endocarditis, diastolic dysfunction, cerebrovascular disease, valve disease, mitral valve prolapse, venous thromboembolism or arrhythmia.

Additionally, methods of the current disclosure include administering fibroblast regenerative cells, a population or plurality or culture of fibroblast regenerative cells, progeny of fibroblast regenerative cells or conditioned medium of fibroblast regenerative cells to treat a subject having or at risk of having a neurological or muscular disease or disorder. Non-limiting examples of neurological and muscular diseases and disorders include multiple sclerosis (MS), spinal cord injury, muscular dystrophy (Becker's or Duchenne's), amyotrophic lateral sclerosis (ALS; Lou Gehrig's disease or classical motor neuron disease), autism, progressive bulbar palsy (progressive bulbar atrophy), pseudobulbar palsy, primary lateral sclerosis (PLS), progressive muscular atrophy, spinal muscular atrophy (SMA, including SMA type I—Werdnig-Hoffmann disease, SMA type II, or SMA type III—Kugelberg-Welander disease), Fazio-Londe disease, Kennedy disease (progressive spinobulbar muscular atrophy), congenital SMA with arthrogryposis, and post-polio syndrome (PPS).

In some embodiments, methods of the current disclosure also comprise administering fibroblast regenerative cells, a population or plurality or culture of fibroblast regenerative cells, progeny of fibroblast regenerative cells or conditioned medium of fibroblast regenerative cells to treat a subject having or at risk of having an immune or inflammatory mediated disorder or disease, such as an autoimmune disease or disorder Non-limiting examples include: Thyroiditis, insulitis, multiple sclerosis, iridocyclitis, uveitis, orchitis, Addison's disease, myasthenia gravis, rheumatoid arthritis, lupus erythematosus, immune hyperreactivity, insulin dependent diabetes mellitus, anemia (aplastic, hemolytic), hepatitis, autoimmune hepatitis, skleritis, idiopathic thrombocytopenic purpura, diseases of the gastrointestinal tract (e.g., Crohn's disease, ulcerative colitis and other inflammatory bowel diseases), juvenile arthritis, scleroderma and systemic sclerosis, sjogren's syndrom, undifferentiated connective tissue syndrome, antiphospholipid syndrome, vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, inflammatory dermatitis, unwanted immune reactions and inflammation associated with arthritis, including rheumatoid arthritis, inflammation associated with hypersensitivity and allergic reactions, systemic lupus erythematosus, collagen diseases, inflammation associated with atherosclerosis, arteriosclerosis, atherosclerotic heart disease, reperfusion injury, vascular inflammatory disorders, respiratory distress syndrome or other cardiopulmonary diseases, inflammation associated with peptic ulcer, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the preventing or treating graft rejection in cases of transplantation of natural or artificial cells, tissue and organs such as liver, kidney, heart, lung, cornea, bone marrow, organs, lenses, pacemakers, natural or artificial skin tissue.

Still further, methods include administering fibroblast regenerative cells, a population or plurality or culture of fibroblast regenerative cells, progeny of fibroblast regenerative cells or conditioned medium of fibroblast regenerative cells to a subject in need of stimulating, increased, inducing, augmenting, or enhanced immunological tolerance. Such methods can stimulate, increase, induce, augment, or enhance immunological tolerance thereby treating an autoimmune disorder.

Still moreover, methods of the current disclosure include administering fibroblast regenerative cells, a population or plurality or culture of fibroblast regenerative cells, progeny of fibroblast regenerative cells or conditioned medium of fibroblast regenerative cells to a subject in need of inhibiting, reducing, decreasing, blocking, preventing, controlling or limiting immunological rejection of a transplant, transplant fibrosis or graft failure. Such methods can inhibit, reduce, decrease, block, prevent, control or limit immunological rejection of the transplant, transplant fibrosis or graft failure thereby enhancing acceptance of the transplant or graft by the subject.

Additionally, methods of the current disclosure include administering fibroblast regenerative cells, a population or plurality or culture of fibroblast regenerative cells, progeny of fibroblast regenerative cells or conditioned medium of fibroblast regenerative cells to treat a subject in need of treatment for a melanoma.

Fibroblast regenerative cells can be administered or delivered to a subject by any route suitable for the treatment method or protocol. Specific non-limiting examples of administration and delivery routes include parenteral, e.g., intravenous, intramuscular, intrathecal (intra- spinal), intrarterial, intradermal, subcutaneous, intra-pleural, transdermal (topical), transmucosal, intra-cranial, intra-ocular, mucosal, implantation and transplantation.

The fibroblast regenerative cells of the current disclosure can be autologous with respect to the subject, that is, the stem cells used in the method (or to produce the conditioned medium) were obtained or derived from a cell from the subject that is treated according to the method. Fibroblast regenerative cells, a population or plurality or culture of fibroblast regenerative cells, progeny of fibroblast regenerative cells or conditioned medium of fibroblast regenerative cells can be allogeneic with respect to the subject, that is, the stem cells used in the method (or to produce the conditioned medium) were obtained or derived from a cell from a subject that is different from the subject that is treated according to the method.

In one embodiment of the disclosure, disclosed is a fibroblast population and various fibroblast populations that are capable of proliferating and differentiating into at least two of ectoderm, mesoderm, or endoderm, are plastic adherent, possess an enhanced proliferative activity as compared to non-selected fibroblasts, and express one or more of the following genes: Oct-4, KLF-4, Nanog, CD117, Sox-2, CD34 ,Rex-1, GDF-3, CD105, IL-10, and Stella. In another embodiment, the cells are cryopreserved and the population is included within a container (e.g., vial, syringe or other container suitable for local delivery into a site within a human or animal, such as a bag or other container suitable for intravenous delivery of cells within a human or animal). In another embodiment, the population comprises stem cells in an amount of at least 1×10², 1×10⁶, 1×10⁹, 1×10¹⁰, 1×10¹², 1×10₁₄. In some embodiments, the amount are 1>10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹ , 1×10²⁰, 1×10²⁵, 1×10-, 1×10³⁵, 1×10⁴⁰, 1×10⁴⁵, 1×10⁵⁰, 1×10⁵⁵, 1×10⁶⁰, 1×10⁶⁵, 1×10⁷⁰, 1×10⁷⁵, 1×1080, 1×10⁸⁵, 1×10⁹⁰, 1×1095, 1×10¹⁰⁰, or any amounts in between

In another embodiment, the population is contained in a 0.9% NaC1 solution. In one embodiment, the population further contains a bioactive compound (e.g., expresses a growth factor, a cytokine, an antibody or fragment thereof, or the population contains an organic molecule having a mass of less than 5,000 daltons. In another aspect, the invention provides a master cell bank containing a plurality of cryopreserved individually packaged populations of isolated adult stem cells, each population including at least 1×102 or more of the cells of previous aspect. The cells derived by the invention are capable of differentiating into various tissues, for example, the invention provides a method of forming a neural cell, the method involving the steps of contacting a fibroblast selected/cultured for enhanced regenerative activity under neural cell-forming conditions. In one embodiment, the conditions include culturing the cell with bFGF, FGF-8, SHH, and BDNF. In another aspect, the invention provides a method of forming a hepatocyte, the method involving culturing a regenerative cell derived from fibroblasts under hepatocyte-forming conditions. In one embodiment, the conditions include culturing the cell with hepatocyte growth factor (HGF) and FGF-4. In another embodiment of the disclosure, provided is a method of forming an endothelial cell, the method involving culturing a regenerative fibroblast cell under endothelial cell-forming conditions. In one embodiment, the conditions include conditions include culturing the cell with VEGF.

In another embodiment, provided herein is a method of forming a hematopoietic cell, the method involving culturing a fibroblast regenerative cell under hematopoietic cell forming conditions. In one embodiment, the conditions include conditions include culturing the cell with bone morphogenic protein-4 (BMP4), VEGF, bFGF, stem cell factor (SCF), Flt3L, hyper IL6, thrombopoietin (TPO) and erythropoietin (EPO).

In another embodiment of the disclosure, means of treating a cardiovascular disease in a subject are disclosed, the method involving administering to the subject a fibroblast regenerative cell, or a committed or differentiated progeny thereof, in an amount sufficient to treat the disease. In one embodiment, the cardiovascular disease is myocardial infarction, congestive heart failure, ischemic cardiomyopathy, and coronary artery disease. In another aspect, the invention provides a method of increasing vascularization in a subject, the method involving administering to the subject a fibroblast derived regenerative cell, or a committed or differentiated progeny thereof, in an amount sufficient to increase vascularization. In one embodiment, the subject is suffering from type II diabetes. In another embodiment the patient is suffering of cardiovascular disease. In another aspect, the invention provides a method for treating a neurological disorder in a subject, the method involving administering to the subject a fibroblast derived regenerative cell or a committed or differentiated progeny thereof, in an amount sufficient to treat the disease. In one embodiment, the neurological disorder is a neurodegenerative disease (e.g., Parkinson's disease, Alzheimer's disease, or Huntington's disease) or neurological injury.

In another embodiment, the disclosure provides a process for expanding the population of fibroblast regenerative cells involving passaging the population at least about three, four, five, six, seven, eight, nine, ten, fifteen, twenty, thirty, or forty times. In another embodiment, the invention provides a process for differentiating the isolated fibroblast regenerative cell under conditions sufficient to differentiate the isolated stem cell.

In various embodiments of the above aspects or any other aspect of the disclosure delineated herein, the antigens used as the basis of selection are CD105, CD117 and/or CD34. In other embodiments, the cell is substantially purified (e.g., at least about 20%, 25%, 30%, 40%, 50%, 75%, 80% or more cells of the invention. In other embodiments, the cell is isolated from a mammal (e.g., human). In other embodiments, the cell is isolated from an adult mammal. In still other embodiments, the cell contains a heterologous nucleic acid sequence.

In another embodiment, the disclosure provides an isolated (e.g., purified or substantially purified) fibroblast regenerative cell capable of proliferating and differentiating into ectoderm, mesoderm, and endoderm, expresses at least one of (e.g., at least 2, 4, 5, or 6 of) Oct-4, Nanog, Sox-2, KLF4, c-Myc, Rex-1, GDF-3, LIF receptor, CD105, CD117, CD344, IL-10, and Stella, and does not express at least one of (e.g., at least 2, 3, 4, 5, 6, 7, 8, or all of) MHC class I, MHC class II, CD45, CD13, CD49c, CD66b, and CD90 cell surface proteins. In another aspect, the disclosure features a population of cells including cells of either of the previous two aspects. The population may contain at least 5%, 10%, 20%, 30%, 40%, 50%, 60%, 70%, 80%, 90%, 95%, or 99% of the cells of the previous two aspects (e.g., an embryoid body). The population may further be part of a composition including a cryoprotectant (e.g., any described herein). In another embodiment, the population of cells may be part of a composition with stem cells (e.g., any known in the art such as mesenchymal cells or embryonic stem cells) or differentiated cells (e.g., any described herein, including myocytes, adipocytes, ectodermal cells, muscle cells, osteoblasts, chondrocytes, endothelial cells, fibroblasts, pancreatic cells, hepatocytes, bile duct cells, bone marrow cells, neural cells, and genitourinary cells. Such cells may be autologous or allogeneic to the stem cells of the invention.

In another aspect, the disclosure features a method for isolating a population of regenerative fibroblast cells. The method includes the steps (a) providing a tissue with regenerative activity; (b) enriching for a population of cells that are about 6-12 micrometers in size; and may optionally include (c) depleting cells from the population expressing stem cell surface markers or MHC proteins (e.g., any described herein), thereby isolating a population of stem cells. Step (c) may include depleting cells expressing MHC class I, CD66b, glycophorin a, or glycophorin b. The method may further include (d) cryopreserving the cells. In another embodiment, the method further includes (d) transfecting the cells with a polynucleotide vector containing a stem cell-specific promoter (e.g., an Oct-4, Nanog, Sox-9, GDF3, Rex-1, or Sox-2 promoter, or any promoter described herein) operably linked to a reporter or selection gene; and (e) further enriching the population for the regenerative fibroblast cells using expression of the reporter or selection gene (e.g., using flow cytometry). In another embodiment, the method further includes (d) contacting the cells with a detectable compound (e.g., carboxyfluorescein diacetate, succinimidyl ester, or Aldefluor) which enters said cells, the compound being selectively detectable in proliferating and non-proliferating cells; and (e) enriching the population of cells for the proliferating cells. In another embodiment, the method further includes culturing the cells under conditions which form tissue aggregate bodies. The method may further include separating (e.g., by cell depletion) cell types such as granulocytes, T-cells, B-cells, NK-cell, red blood cells, or any combination thereof, from the fibroblast regenerative cells of the invention. The method may further include culturing the population of fibroblast regenerative cells under conditions which support proliferation of the cells. In any of the embodiments of this aspect of the invention, the cells may further be cryopreserved.

The disclosure also features a cell produced by any of the above methods. The invention also provides a method for identifying a fibroblast regenerative cell, the method including the steps of introducing into a cell a vector comprising a cell-specific promoter coupled to at least one selectable marker gene, wherein the fibroblast regenerative cell that does not express at least one of (e.g., at least 2, 3, 4, 5, 6, 7, 8, or all of) MHC class I, MHC class II, CD44, CD45, CD13, CD34, CD49c, CD66b, CD73, CD105, and CD90 cell surface proteins, is capable of differentiating into mesoderm, ectoderm, and endoderm; and expresses at least one embryonic transcription factor (e.g., Oct-4, Nanog, Sox-2, Rex-1, GDF-3, Stella, FoxD3, Polycomb), expressing the selectable marker gene from the cell specific promoter in said stem cell; and detecting expression of the marker gene in the stem cell, thereby identifying the cell. The method may further comprise isolating the fibroblast regenerative cell. The fibroblast cell can be derived from the bodily fluid of a mammal, such as synovial fluid or blood, as well as tissue. Preferably, the mammal is a human. In certain embodiments, the cell does not express CD13, CD44, and CD90.

In various embodiments, the fibroblast cell-specific promoter is an Oct-4 promoter, Nanog promoter, Sox-2 promoter, Rex-1 promoter, GDF-3 promoter, Stella promoter, FoxD3 promoter, Polycomb Repressor Complex 2 promoter, or CTCF promoter. In one embodiment, the fibroblast cell-specific promoter is flanked by loxP sites. In another embodiment, the vector is a retroviral vector. In yet another embodiment, the selectable marker gene encodes a fluorescent protein, such as a Green Fluorescent Protein (GFP). In some embodiments, enhancement of regenerative activity is provided by transfection of fibroblasts with OCT-4 as described for mesenchymal stem cells but applied to fibroblasts (Wang, K. H., et al., Upregulation of Nanog and Sox-2 genes following ectopic expression of Oct-4 in amniotic fluid mesenchymal stem cells. Biotechnol Appl Biochem, 2015. 62(5): p. 591-7, incorporated herein by reference).

In other embodiments, cells of pluripotent ability are fused with fibroblasts to artificially generate fibroblasts with enhanced regenerative activity. Fusion of fibroblast regenerative cells with other cells are described in this reference, which the inventors seek to provide as guidance for application to fibroblasts to endow enhanced regenerative activity (Xu, D., et al., Coculturing embryonic stem cells with damaged hepatocytes leads to restoration of damage and high frequency of fusion. Cell Mol Biol (Noisy-le-grand), 2009. 55 Suppl: p. OL1186-99, incorporated herein by reference).

In other embodiments, transfection with cell permeable NANOG [53], is applied to fibroblasts that are initially selected for expression of CD105 and/or CD117 (such as in Peitz, M., et al., Cell-permeant recombinant Nanog protein promotes pluripotency by inhibiting endodermal specification. Stem Cell Res, 2014. 12(3): p. 680-9, incorporated herein by reference).

In yet another embodiment, a vector is utilized that comprises two selectable marker genes. In a specific embodiment, the two selectable marker genes are a fluorescent protein and a protein sensitive to drug selection. In yet another embodiment, the selectable marker gene encodes a cell surface protein. In another aspect, the invention provides a fibroblast regenerative cell isolated by a method comprising the steps of introducing into a population of fibroblasts a vector comprising a regenerative cell-specific promoter coupled to at least one selectable marker gene, wherein said regenerative cell does not express MHC class I, MHC class II, CD44, CD45, CD13, CD34, CD49c, CD73, CD105 and CD90 cell surface proteins; expressing the selectable marker gene from the regenerative-cell specific promoter in said fibroblast population; and detecting expression of the marker gene in the regenerative fibroblast cell.

In one embodiment, the disclosure provides a method for the differentiation of a fibroblast regenerative cell of the invention into a cell lineage of a germ layer selected from the group consisting of ectoderm, mesoderm and endoderm, and/or a specific cell type including but not limited to a neural, glial, chondroblast, osteoblast, adipocyte, hepatocyte, muscle cell (e.g., smooth muscle or skeletal muscle), cardiac cell, pancreatic cell, pulmonary cell, and endothelial cell.

In some embodiments of the disclosure, fibroblast regenerative cells are administered together with one or more anti-inflammatory agents. Pharmaceutical agents possessing anti-inflammatory properties include Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; Zomepirac Sodium.

In some embodiments, pharmaceutical formulations comprising fibroblast regenerative cells are provided. Pharmaceutical formulations and delivery systems appropriate for the compositions and methods of the invention are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315.

### VI. Kits

Certain aspects of the present disclosure also concern kits containing compositions of the disclosure or compositions to implement methods of the disclosure. In some embodiments, kits can be used to provide fibroblast regenerative cells, population thereof, progeny thereof or conditioned media thereof. In some cases, kits include one or more reagents for producing and/or identifying fibroblast regenerative cells.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

In certain aspects, negative and/or positive control agents are included in some kit embodiments. The control molecules can be used to verify the enhance regenerative activity of fibroblast cells.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

### EXAMPLES

The following examples are included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### Example 1

### Enhanced Expression of OCT-4 in CD105 Purified Fibroblasts

Fibroblasts derived from foreskin were obtained from American Type Culture Collection (ATCC) and grown in Optimem media with 10% fetal calf serum. Isolation of CD105 positive and negative cells was performed using magnetic activated cell sorting (MACS).

Briefly, suspensions of the fibroblasts were obtained from cultured cells by trypsinization. Cells were washed once with 1× PBS and resuspended with (MACS) buffer (1× PBS containing 0.5% fetal bovine serum (FBS; cat. no. SH30087.01; HyClone; GE Healthcare Life Sciences, Logan, Utah, USA) and 2 mM ethylenediamine tetraacetic acid, pH 7.2). A nylon mesh was used to filter cell suspensions (30-μm pore). The cells were resuspended in MACS buffer at 10⁷ cells per 80 μl, mixed with 20 μl microbeads of directly conjugated mouse anti-human CD105 antibody (1:200; cat. no. MCA1557; Bio-Rad Laboratories, Inc., Hercules, Calif., USA), and incubated at 4° C. for 15 min on a rotator in the dark. Following washing in 1× PBS, the DDFCs-CD105 cells were resuspended in MACS buffer and processed in an LS+/VS+ separation column. The column was removed from the magnetic device, and the cells were flushed out with MACS buffer. The CD105− and CD105+ cells were recovered by centrifugation at 300×g for 10 min at 4° C.

Expression of OCT-4, a marker of stem cell potency, was assessed using flow cytometry for assessment of the intracellular OCT-4 protein. Flow cytometry was performed subsequent to the cells being stained by murine anti-OCT-4 (ab91194, Abcam, Cambridge, Mass.; 1:200 dilution). Secondary antibodies were Allophycocyanin anti-mouse IgG (A-865, Life Technologies, Grand Island, N.Y.; 1:1000),.

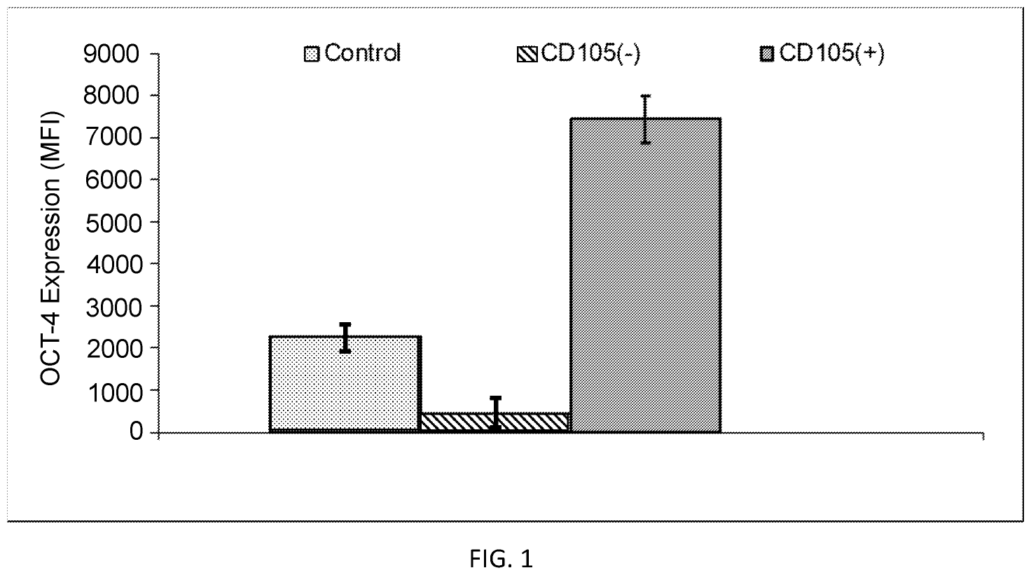

As seen in FIG. 1, expression of OCT-4 was enhanced in cells that have been selected for expression of CD105.

### Example 2

### Enhanced Expression of Nanog in CD105 Purified Fibroblasts

Fibroblasts derived from foreskin were obtained from ATCC and grown in Optimem media with 10% fetal calf serum. Isolation of CD105 positive and negative cells was performed using magnetic activated cell sorting (MACS).

Briefly, suspensions of the fibroblasts were obtained from cultured cells by trypsinization. Cells were washed once with 1× PBS and resuspended with (MACS) buffer (1× PBS containing 0.5% fetal bovine serum (FBS; cat. no. SH30087.01; HyClone; GE Healthcare Life Sciences, Logan, Utah, USA) and 2 mM ethylenediamine tetraacetic acid, pH 7.2). A nylon mesh was used to filter cell suspensions (30-μm pore). The cells were resuspended in MACS buffer at 10⁷ cells per 80 μl, mixed with 20 μl microbeads of directly conjugated mouse anti-human CD105 antibody (1:200; cat. no. MCA1557; Bio-Rad Laboratories, Inc., Hercules, Calif., USA), and incubated at 4° C. for 15 min on a rotator in the dark. Following washing in 1× PBS, the DDFCs-CD105 cells were resuspended in MACS buffer and processed in an LS+/VS+ separation column. The column was removed from the magnetic device, and the cells were flushed out with MACS buffer. The CD105- and CD105+ cells were recovered by centrifugation at 300×g for 10 min at 4° C.

Expression of Nanog, a marker of potency, was assessed using flow cytometry mouse polyclonal antibody against human Nanog purchased from Abcam (Cambridge, Mass.).

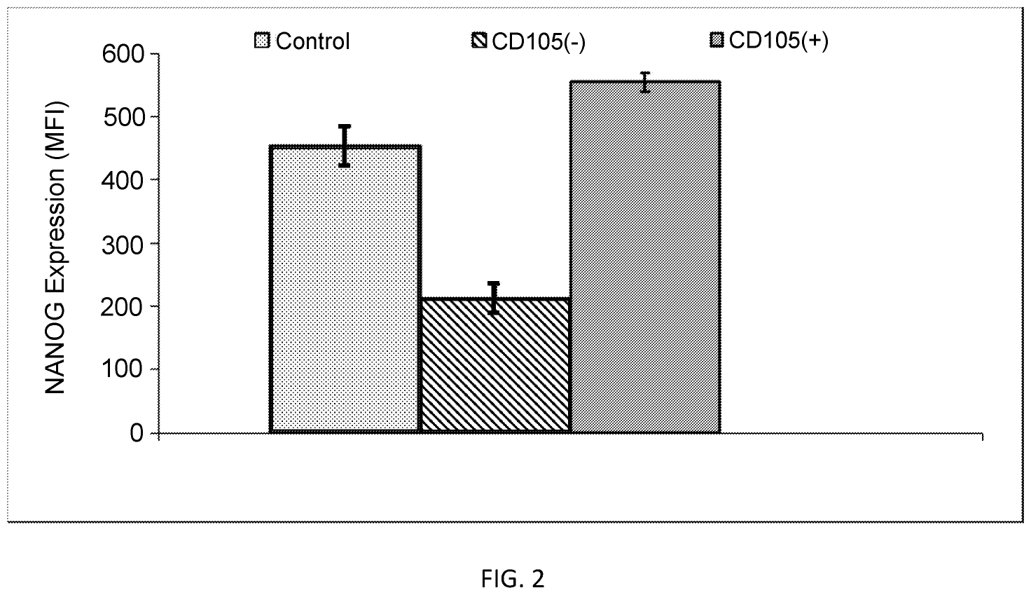

As seen in FIG. 2, expression of NANOG was enhanced in cells that have been selected for expression of CD105.

### Example 3

### Enhanced Expression of KLF-4 in CD105 Purified Fibroblasts

Fibroblasts derived from foreskin were obtained from ATCC and grown in Optimem media with 10% fetal calf serum. Isolation of CD105 positive and negative cells was performed using magnetic activated cell sorting (MACS).

Briefly, suspensions of the fibroblasts were obtained from cultured cells by trypsinization. Cells were washed once with 1× PBS and resuspended with (MACS) buffer (1× PBS containing 0.5% fetal bovine serum (FBS; cat. no. SH30087.01; HyClone; GE Healthcare Life Sciences, Logan, Utah, USA) and 2 mM ethylenediamine tetraacetic acid, pH 7.2). A nylon mesh was used to filter cell suspensions (30-μm pore). The cells were resuspended in MACS buffer at 10⁷ cells per 80 μl, mixed with 20 μl microbeads of directly conjugated mouse anti-human CD105 antibody (1:200; cat. no. MCA1557; Bio-Rad Laboratories, Inc., Hercules, Calif., USA), and incubated at 4° C. for 15 min on a rotator in the dark. Following washing in 1× PBS, the DDFCs-CD105 cells were resuspended in MACS buffer and processed in an LS+/VS+ separation column. The column was removed from the magnetic device, and the cells were flushed out with MACS buffer. The CD105- and CD105+ cells were recovered by centrifugation at 300×g for 10 min at 4° C.

Expression of Nanog, a marker of potency, was assessed using flow cytometry mouse polyclonal antibody against human KLF-4 purchased from Abcam (Cambridge, Mass.).

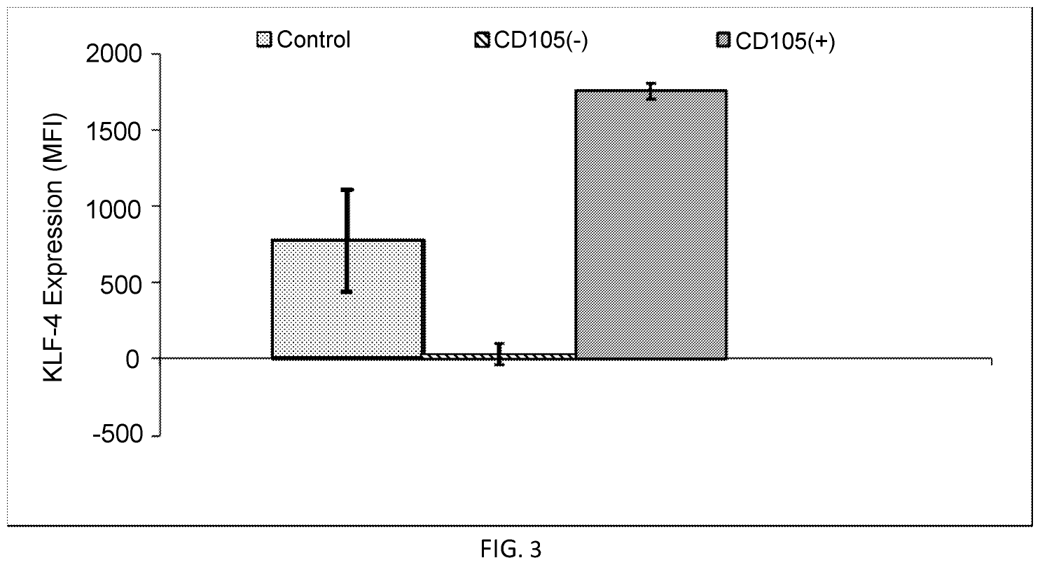

As seen in FIG. 3, expression of KLF-4 was enhanced in cells which have been selected for expression of CD105.

### Example 4

### Enhanced Expression of Immune Modulatory Cytokine IL-10 by CD105 Selected Fibroblasts

Fibroblasts derived from foreskin were obtained from ATCC and grown in Optimem media with 10% fetal calf serum. Isolation of CD105 positive and negative cells was performed using magnetic activated cell sorting (MACS).

Briefly, suspensions of the fibroblasts was obtained from cultured cells by trypsinization. Cells were washed once with 1× PBS and resuspended with (MACS) buffer (1× PBS containing 0.5% fetal bovine serum (FBS; cat. no. SH30087.01; HyClone; GE Healthcare Life Sciences, Logan, Utah, USA) and 2 mM ethylenediamine tetraacetic acid, pH 7.2). A nylon mesh was used to filter cell suspensions (30-μm pore). The cells were resuspended in MACS buffer at 10⁷ cells per 80 μl, mixed with 20 μl microbeads of directly conjugated mouse anti-human CD105 antibody (1:200; cat. no. MCA1557; Bio-Rad Laboratories, Inc., Hercules, Calif., USA), and incubated at 4° C. for 15 min on a rotator in the dark. Following washing in 1× PBS, the DDFCs-CD105 cells were resuspended in MACS buffer and processed in an LS+/VS+ separation column. The column was removed from the magnetic device, and the cells were flushed out with MACS buffer. The CD105− and CD105+ cells were recovered by centrifugation at 300×g for 10 min at 4° C.

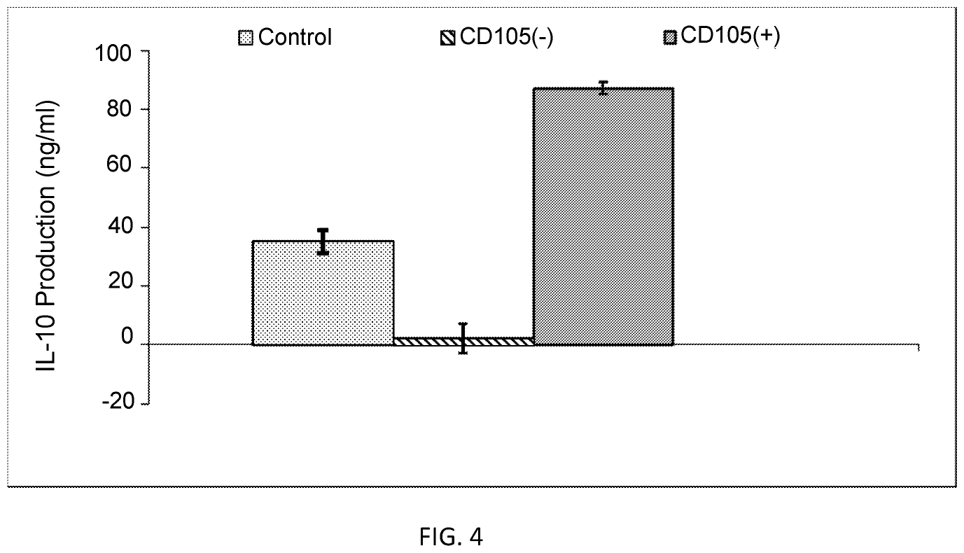

Cells were cultured with allogeneic lymphocytes at a 1:1 ratio for 72 hours and production of IL-10 was assessed by ELISA (R&D Systems). As seen in FIG. 4, an increased production of IL-10 was observed in the CD105 selected fibroblasts.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All publications mentioned in the specification are indicative of the level of those skilled in the art to which the invention pertains. All publications are herein incorporated by reference to the same extent as if each individual publication was specifically and individually indicated to be incorporated by reference.

1. Jossen, V., et al., Manufacturing human mesenchymal stem cells at clinical scale: process and regulatory challenges. Appl Microbiol Biotechnol, 2018. 102(9): p. 3981-3994.

2. Sher, N. and R. Ofir, Placenta-Derived Adherent Stromal Cell Therapy for Hematopoietic Disorders: A Case Study of PLX-R18. Cell Transplant, 2018. 27(1): p. 140-150.

3. Bunpetch, V., et al., From “Bench to Bedside”: Current Advancement on Large-Scale Production of Mesenchymal Stem Cells. Stem Cells Dev, 2017. 26(22): p. 1662-1673.

4. Kozanoglu, I., et al., Quantum cell expansion system: Safe and rapid expansion. Cytotherapy, 2017. 19(10): p. 1246-1247.

5. Rafiq, Q. A., et al., Process development of human multipotent stromal cell microcarrier culture using an automated high-throughput microbioreactor. Biotechnol Bioeng, 2017. 114(10): p. 2253-2266.

6. Dwarshuis, N. J., et al., Cells as advanced therapeutics: State-of-the-art, challenges, and opportunities in large scale biomanufacturing of high-quality cells for adoptive immunotherapies. Adv Drug Deliv Rev, 2017. 114: p. 222-239.

7. Panchalingam, K. M., et al., Bioprocessing strategies for the large-scale production of human mesenchymal stem cells: a review. Stem Cell Res Ther, 2015. 6: p. 225.

8. Robey, P. G., et al., Generation of clinical grade human bone marrow stromal cells for use in bone regeneration. Bone, 2015. 70: p. 87-92.

9. Petrenko, Y. A., et al., Perfusion bioreactor-based cryopreservation of 3D human mesenchymal stromal cell tissue grafts. Cryobiology, 2017. 76: p. 150-153.

10. Barckhausen, C., et al., GMP-Compliant Expansion of Clinical-Grade Human Mesenchymal Stromal/Stem Cells Using a Closed Hollow Fiber Bioreactor. Methods Mol Biol, 2016. 1416: p. 389-412.

11. Simmons, A. D., et al., Sensing metabolites for the monitoring of tissue engineered construct cellularity in perfusion bioreactors. Biosens Bioelectron, 2017. 90: p. 443-449.

12. Lambrechts, T., et al., Large-Scale Mesenchymal Stem/Stromal Cell Expansion: A Visualization Tool for Bioprocess Comparison. Tissue Eng Part B Rev, 2016. 22(6): p. 485-498.

13. Fernandes-Platzgummer, A., et al., Clinical-Grade Manufacturing of Therapeutic Human Mesenchymal Stem/Stromal Cells in Microcarrier-Based Culture Systems. Methods Mol Biol, 2016. 1416: p. 375-88.

14. Neumann, A., et al., Characterization and Application of a Disposable Rotating Bed Bioreactor for Mesenchymal Stem Cell Expansion. Bioengineering (Basel), 2014. 1(4): p. 231-245.

15. Zanetti, A., et al., Suspension-Expansion of Bone Marrow Results in Small Mesenchymal Stem Cells Exhibiting Increased Transpulmonary Passage Following Intravenous Administration. Tissue Eng Part C Methods, 2015. 21(7): p. 683-92.

16. Elseberg, C. L., D. Salzig, and P. Czermak, Bioreactor expansion of human mesenchymal stem cells according to GMP requirements. Methods Mol Biol, 2015. 1283: p. 199-218.

17. Tsai, A. C. and T. Ma, Expansion of Human Mesenchymal Stem Cells in a Microcarrier Bioreactor. Methods Mol Biol, 2016. 1502: p. 77-86.

18. Petry, F., et al., Manufacturing of Human Umbilical Cord Mesenchymal Stromal Cells on Microcarriers in a Dynamic System for Clinical Use. Stem Cells Int, 2016. 2016: p. 4834616.

19. Sart, S. and S. N. Agathos, Large-Scale Expansion and Differentiation of Mesenchymal Stem Cells in Microcarrier-Based Stirred Bioreactors. Methods Mol Biol, 2016. 1502: p. 87-102.

20. Rafiq, Q. A., et al., Systematic microcarrier screening and agitated culture conditions improves human mesenchymal stem cell yield in bioreactors. Biotechnol J, 2016. 11(4): p. 473-86.

21. Wang, X., et al., Perfusion culture-induced template-assisted assembling of cell-laden microcarriers is a promising route for fabricating macrotissues. Biotechnol J, 2014. 9(11): p. 1425-34.

22. Carmelo, J. G., et al., Scalable ex vivo expansion of human mesenchymal stem/stromal cells in microcarrier-based stirred culture systems. Methods Mol Biol, 2015. 1283: p. 147-59.

23. Hupfeld, J., et al., Modulation of mesenchymal stromal cell characteristics by microcarrier culture in bioreactors. Biotechnol Bioeng, 2014. 111(11): p. 2290-302.

24. Tozetti, P. A., et al., Expansion strategies for human mesenchymal stromal cells culture under xeno-free conditions. Biotechnol Prog, 2017. 33(5): p. 1358-1367.

25. Mizukami, A., et al., Stirred tank bioreactor culture combined with serum-xenogeneic-free culture medium enables an efficient expansion of umbilical cord-derived mesenchymal stem/stromal cells. Biotechnol J, 2016. 11(8): p. 1048-59.

26. Carmelo, J. G., et al., A xeno-free microcarrier-based stirred culture system for the scalable expansion of human mesenchymal stem/stromal cells isolated from bone marrow and adipose tissue. Biotechnol J, 2015. 10(8): p. 1235-47.

27. Nold, P., et al., Immunosuppressive capabilities of mesenchymal stromal cells are maintained under hypoxic growth conditions and after gamma irradiation. Cytotherapy, 2015. 17(2): p. 152-62.

28. Wang, J., et al., Stem cell-derived mitochondria transplantation: a novel strategy and the challenges for the treatment of tissue injury. Stem Cell Res Ther, 2018. 9(1): p. 106.

29. Caicedo, A., et al., Artificial Mitochondria Transfer: Current Challenges, Advances, and Future Applications. Stem Cells Int, 2017. 2017: p. 7610414.

30. Melcher, M., et al., Modulation of oxidative phosphorylation and redox homeostasis in mitochondrial NDUFS4 deficiency via mesenchymal stem cells. Stem Cell Res Ther, 2017. 8(1): p. 150.

31. Morrison, T. J., et al., Mesenchymal Stromal Cells Modulate Macrophages in Clinically Relevant Lung Injury Models by Extracellular Vesicle Mitochondrial Transfer. Am J Respir Crit Care Med, 2017. 196(10): p. 1275-1286.

32. Li, C. J., et al., Enhancement of Mitochondrial Transfer by Antioxidants in Human Mesenchymal Stem Cells. Oxid Med Cell Longev, 2017. 2017: p. 8510805.

33. Jackson, M. V. and A. D. Krasnodembskaya, Analysis of Mitochondrial Transfer in Direct Co-cultures of Human Monocyte-derived Macrophages (MDM) and Mesenchymal Stem Cells (MSC). Bio Protoc, 2017. 7(9).

34. Nzigou Mombo, B., et al., MitoCeption: Transferring Isolated Human MSC Mitochondria to Glioblastoma Stem Cells. J Vis Exp, 2017(120).

35. Reznichenko, A. S., C. Huyser, and M. S. Pepper, Mitochondrial transfer: Implications for assisted reproductive technologies. Appl Transl Genom, 2016. 11: p. 40-47.

36. Kang, E., et al., Mitochondrial replacement in human oocytes carrying pathogenic mitochondrial DNA mutations. Nature, 2016. 540(7632): p. 270-275.

37. Wu, T. H., et al., Mitochondrial Transfer by Photothermal Nanoblade Restores Metabolite Profile in Mammalian Cells. Cell Metab, 2016. 23(5): p. 921-9.

38. Plotnikov, E. Y., et al., Intercellular Transfer of Mitochondria. Biochemistry (Mosc), 2015. 80(5): p. 542-8.

39. Paliwal, S., et al., Regenerative abilities of mesenchymal stem cells through mitochondrial transfer. J Biomed Sci, 2018. 25(1): p. 31.

40. Rodriguez, A. M., et al., Intercellular mitochondria trafficking highlighting the dual role of mesenchymal stem cells as both sensors and rescuers of tissue injury. Cell Cycle, 2018: p. 1-25.

41. Babenko, V. A., et al., Mirol Enhances Mitochondria Transfer from Multipotent Mesenchymal Stem Cells (MMSC) to Neural Cells and Improves the Efficacy of Cell Recovery. Molecules, 2018. 23(3).

42. Wang, J., et al., Cell adhesion-mediated mitochondria transfer contributes to mesenchymal stem cell-induced chemoresistance on T cell acute lymphoblastic leukemia cells. J Hematol Oncol, 2018. 11(1): p. 11.

43. Guo, R., D. Davis, and Y. Fang, Intercellular transfer of mitochondria rescues virus-induced cell death but facilitates cell-to-cell spreading of porcine reproductive and respiratory syndrome virus. Virology, 2018. 517: p. 122-134.

44. Wang, Z. B., et al., Transfer of autologous mitochondria from adipose tissue-derived stem cells rescues oocyte quality and infertility in aged mice. Aging (Albany NY), 2017. 9(12): p. 2480-2488.

45. Li, X., et al., Mesenchymal stem cells alleviate oxidative stress-induced mitochondrial dysfunction in the airways. J Allergy Clin Immunol, 2017.

46. Chuang, Y. C., et al., Mitochondrial Transfer from Wharton's Jelly Mesenchymal Stem Cell to MERRF Cybrid Reduces Oxidative Stress and Improves Mitochondrial Bioenergetics. Oxid Med Cell Longev, 2017. 2017: p. 5691215.

47. Jiang, D., et al., Mitochondrial transfer of mesenchymal stem cells effectively protects corneal epithelial cells from mitochondrial damage. Cell Death Dis, 2016. 7(11): p. e2467.

48. Jackson, M. V., et al., Mitochondrial Transfer via Tunneling Nanotubes is an Important Mechanism by Which Mesenchymal Stem Cells Enhance Macrophage Phagocytosis in the In vitro and In vivo Models of ARDS. Stem Cells, 2016. 34(8): p. 2210-23.

49. Yang, H., et al., Biochip-based study of unidirectional mitochondrial transfer from stem cells to myocytes via tunneling nanotubes. Biofabrication, 2016. 8(1): p. 015012.

50. Han, H., et al., Bone marrow-derived mesenchymal stem cells rescue injured H9c2 cells via transferring intact mitochondria through tunneling nanotubes in an in vitro simulated ischemia/reperfusion model. Mol Med Rep, 2016. 13(2): p. 1517-24.

51. Wang, K. H., et al., Upregulation of Nanog and Sox-2 genes following ectopic expression of Oct-4 in amniotic fluid mesenchymal stem cells. Biotechnol Appl Biochem, 2015. 62(5): p. 591-7.

52. Xu, D., et al., Coculturing embryonic stem cells with damaged hepatocytes leads to restoration of damage and high frequency of fusion. Cell Mol Biol (Noisy-le-grand), 2009. 55 Suppl: p. OL1186-99.

53. Peitz, M., et al., Cell-permeant recombinant Nanog protein promotes pluripotency by inhibiting endodermal specification. Stem Cell Res, 2014. 12(3): p. 680-9.

What is claimed is:

1. A method of treating a medical condition in a subject, comprising administering to the subject a composition comprising an isolated fibroblast regenerative cell, a population thereof, progeny thereof and/or a conditioned medium thereof, wherein the fibroblast regenerative cell has an increased regenerative activity compared to a control fibroblast cell.

2. The method of claim 1, wherein the isolated fibroblast regenerative cell or population thereof expresses CD105 marker and/or CD117 marker.

3. The method of claim 1 or 2, wherein the fibroblast regenerative cell further comprises a rhodamine 123 efflux activity.

4. The method of any of claims 1-3, wherein the fibroblast regenerative cell expresses at least one additional marker selected from the group consisting of Oct-4, CD-34, KLF-4, Nanog, Sox-2, Rex-1, GDF-3, IL-10, Stella, and a combination thereof.

5. The method of any of claims 1-4, wherein the fibroblast regenerative cell has enhanced expression of GDF-11 as compared to a control cell.

6. The method of any of claims 1-5, wherein the fibroblast regenerative cell is derived from a tissue having regenerative properties.

7. The method of any of claims 1-6, wherein the fibroblast regenerative cell is derived from placental tissue, umbilical cord tissue, endometrial cells, Wharton's jelly, bone marrow or adipose tissue.

8. The method of any of claims 1-7, wherein the further comprises a step of adding exogenous mitochondria to the composition.

9. The method of claim 8, wherein the exogenous mitochondria are isolated and substantially pure from other cellular components.

10. The method of any of claims 8-9, wherein the mitochondria are administered to the fibroblast regenerative cells by lipid fusion, polyethylene glycol-mediated fusion or by electroporation.

11. The method of any of claims 8-10, wherein the mitochondria are encapsulated or treated with agents to facilitate the incorporation of the mitochondria into the fibroblast cell.

12. The method of any of claims 1-11, wherein the composition further includes vitamin A, vitamin C, vitamin D, vitamin E, vitamin K, folic acid, choline, vitamin B1, vitamin B2, vitamin B5, vitamin B6, vitamin B12, biotin, nicotinamide, betacarotene, coenzyme Q, selenium, superoxide dismutase, glutathione peroxide, uridine, creatine succinate, pyruvate, dihydroxyacetone), acetyl-L-carnitine, alpha-lipoic acid, cardiolipin, omega fatty acid, lithium carbonate, lithium citrate, calcium, or any combination thereof.

13. The method of any of claims 8-12, wherein the mitochondria are autologous, syngeneic mitochondria, allogeneic mitochondria, or xenogeneic mitochondria.

14. The method of any of claims 8-13, wherein the mitochondria are derived from stem cells, or from cells less differentiated as compared to the fibroblast regenerative cell.

15. The method of any of claims 1-14, wherein the method further includes the step of administering isolated and substantially pure mitochondria into the subject.

16. The method of claim 1-15, wherein the method further includes the steps of i) separating mitochondria from other constituents of a cell to produce isolated and substantially pure mitochondria; and (ii) administering the isolated and substantially pure mitochondria into the subject.

17. The method of any of claims 1-16, wherein the regenerative fibroblast cell is capable of proliferating and differentiating into at least two of ectoderm, mesoderm or endoderm.

18. The method of any one of claims 1-17, wherein the condition is an immune disorder, a muscular disorder, a hematopoietic disorder, a liver disorder, an angiogenesis disorder, a pancreatic disorder, a cardiac disease, a pulmonary disease, neurological disease, neurological disorder, neurodegenerative disease, muscular disorder, muscular disease, immune disease, inflammatory-mediated disease, inflammatory-mediated disorder, inflammation, ischemia, stroke, schemic heart disease, liver failure, kidney failure, peripheral artery disease, pulmonary fibrosis, liver fibrosis, pancreatic fibrosis, diabetic limb, fibrosis, scar tissue formation, pathological apoptosis, diabetes, cirhossis, hepatitis, osteoporosis, a bone fracture, a neurological injury, the need for a prosthesis in a joint, the need for dermal stem cells, the need to increase the proliferation of islet cells, the need to increase proliferation of hepatocytes, the need to increase insulin production, the need for osteocytes, the need to increase osteocyte formation, the need to increase osteocyte function or the need of cell therapy.

19. The method of claim 18, wherein the cardiac disease or pulmonary disease is atherosclerosis, myocardial infarction (Heart Attack), cardiac infection, heart failure, ischemic heart failure, high blood pressure (Hypertension), or pulmonary hypertension, idiopathic pulmonary fibrosis, stroke, congenital heart disease (CHD), congestive heart failure, angina, myocarditis, coronary artery disease, cardiomyopathy, dilated cardiomyopathy, hypertrophic cardiomyopathy, endocarditis, diastolic dysfunction, cerebrovascular disease, valve disease, mitral valve prolapse, venous thromboembolism or arrhythmia.

20. The method of claim 18, wherein the neurological or muscular disease is multiple sclerosis (MS), spinal cord injury, muscular dystrophy (Becker's or Duchenne's), amyotrophic lateral sclerosis (ALS; Lou Gehrig's disease or classical motor neuron disease), autism, progressive bulbar palsy (progressive bulbar atrophy), pseudobulbar palsy, primary lateral sclerosis (PLS), progressive muscular atrophy, spinal muscular atrophy (SMA, including SMA type I—Werdnig-Hoffmann disease, SMA type II, or SMA type III—Kugelberg-Welander disease), Fazio-Londe disease, Kennedy disease (progressive spinobulbar muscular atrophy), congenital SMA with arthrogryposis, and post-polio syndrome (PPS).

21. The method of claim 18, wherein the immune disorder or the inflammatory-mediated disorder or the immune disease or the inflammatory-mediated disease is thyroiditis, insulitis, multiple sclerosis, iridocyclitis, uveitis, orchitis, Addison's disease, myasthenia gravis, rheumatoid arthritis, lupus erythematosus, immune hyperreactivity, insulin dependent diabetes mellitus, anemia, aplastic anemia, hemolytic anemia, hepatitis, autoimmune hepatitis, skleritis, idiopathic thrombocytopenic purpura, auto immune diseases, diseases of the gastrointestinal tract, Crohn's disease, ulcerative colitis, inflammatory bowel diseases, juvenile arthritis, scleroderma and systemic sclerosis, Sjogren's syndrome, undifferentiated connective tissue syndrome, antiphospholipid syndrome, vasculitis, polyarteritis nodosa, allergic granulomatosis, angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans, polymyalgia rheumatica, essentiell cryoglobulinemia, Psoriasis vulgaris and psoriatic arthritis, diffuse fasciitis with eosinophilia, diffuse fasciitis without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, inflammatory dermatitis, unwanted immune reactions and inflammation associated with arthritis, rheumatoid arthritis, inflammation associated with hypersensitivity and allergic reactions, systemic lupus erythematosus, collagen diseases, inflammation associated with atherosclerosis, arteriosclerosis, atherosclerotic heart disease, reperfusion injury, vascular inflammatory disorders, respiratory distress syndrome, cardiopulmonary diseases, inflammation associated with peptic ulcer, hepatic fibrosis, liver cirrhosis, hepatic diseases, thyroiditis or other glandular diseases, glomerulonephritis, renal diseases, urologic diseases, otitis, oto-rhino-laryngological diseases, dermatitis, dermal diseases, periodontal diseases, dental diseases, orchitis or epididimo-orchitis, infertility, orchidal trauma, immune related testicular diseases, placental dysfunction, placental insufficiency, habitual abortion, eclampsia, pre-eclampsia, immune-related gynaecological diseases, inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, immune reaction to ocular implants, inflammation reaction against ocular implants, immune-related ophthalmic diseases, inflammatory-related ophthalmic diseases, inflammation associated with autoimmune diseases or conditions or disorders of the central nervous system (CNS) or in any other organ, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, pseudo-tumour cerebri, Down's Syndrome, Huntington's disease, amyotrophic lateral sclerosis, inflammatory components of CNS compression, inflammatory components of CNS compression, inflammatory components of CNS compression, CNS trauma infections of the CNS, inflammatory components of muscular atrophies, inflammatory components of muscular dystrophies, conditions or disorders of the central and peripheral nervous systems, post-traumatic inflammation, septic shock, infectious diseases, inflammatory complications of surgery, inflammatory complications of organ transplant, side effects of surgery, side effects of organ transplants, inflammatory complications of gene therapy, immune complications of gene therapy, inflammatory-related side effects of gene therapy, immune-related side effects of gene therapy, inflammation associated with AIDS, humoral immune response, cellular immune response, monocyte proliferative disease, leukocyte proliferative diseases, leukemia, high amounts of monocytes or lymphocytes, or graft rejection.

22. The method of claim 21, wherein the graft rejection is after transplantation of natural cells, artificial cells, a natural tissue, an artificial tissue, bone marrow, an organ, a pacemaker, a cornea or a lens.

23. The method of claim 22, wherein the organ is a liver, a kidney, a heart, or a lung.

24. The method of claim 18, wherein the neurodegenerative disease is Parkinson's disease, Alzheimer's disease, or Huntington disease.

25. The method of any one of claims 1-24, wherein the subject is in need of increased hematopoiesis, increased liver activity, increased angiogenesis, controlled inflammation, controlled autoimmunity.

26. The method of any one of claims 1-25, wherein the subject has ischemia.

27. The method of claim 26, wherein the ischemia is in a cardiac tissue, a pulmonary tissue, a kidney tissue, or a limb.

28. The method of claim 26 or 27, wherein the ischemia is associated with stroke, ischemic heart disease, liver failure, kidney failure, and peripheral artery disease.

29. The method of any one of claims 1-28, wherein the subject has or is at risk of developing a stroke, pulmonary fibrosis, a diabetic limb, an ischemic heart disease, liver failure, kidney failure, peripheral artery disease, diabetes, liver failure, cirrhosis, liver or pancreas fibrosis, or hepatitis, osteoporosis, bone fracture, cardiac disease, or pulmonary disease.

30. The method of claim 29, wherein the cardiac or pulmonary disease is artherosclerosis, myocardial infarction, cardiac infection, heart failure, ischemic heart failure, hypertension, pulmonary hypertension, idiopathic pulmonary fibrosis, stroke, congenital heart disease (CHD), congestive heart failure, angina, myocarditis, coronary artery disease, cardiomyopathy, dilated cardiomyopathy, hypertrophic cardiomyopathy, endocarditis, diastolic dysfunction, cerebrovascular disease, valve disease, mitral valve prolapse, venous thromboembolism or arrhythmia.

31. The method of any one of claims 1-30, wherein the subject has or is at risk of developing fibrosis or scar tissue formation.

32. The method of claim 31, wherein the scar tissue formation or fibrosis is in a pancreatic tissue, a liver tissue, a cardiac tissue, a pulmonary tissue, a limb, liver, pancreas or kidney.

33. The method of any of claims 1-32, wherein the subject is in need of inhibition, reduction, decreased, controlled or reversal of pathological apoptosis.

34. The method of any of claims 1-33, wherein the subject is in need of improved pancreas function, liver function, osteocyte function, insulin production, pulmonary function, cardiac function or a prosthesis in a joint.

35. The method of any of claims 1-34, wherein the subject is in need of an increased number of islet cells, hepatocytes, increased insulin production; an increased number of osteocytes.

36. The method of any of claims 1-35, wherein the subject is in need of dermal stem cells, or activation of endogenous dermal stem cells.

37. The method of any of claims 1-36, wherein the subject is also administered one or more anti-inflammatory agents in addition to the composition.

38. The method of claim 37, wherein the anti-inflammatory agent is administered concurrently with the composition.

39. The method of claim 37, wherein the anti-inflammatory agent is administered before and/or after the composition.

40. The method of any of claims 37-39, wherein the anti-inflammatory agent is Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; Zomepirac Sodium.

41. The method of any of claims 1-40, wherein the composition further comprises a bioactive compound.

42. The method of claim 41, wherein the bioactive compound is a growth factor, a cytokine, an antibody, an antibody fragment, an organic molecule of a mass of less than 5000 daltons.

43. The method of any of claims 1-42, wherein the composition is introduced to the subject parenterally, transdermally, transmucosally, by implantation or by transplantation.

44. The method of claim 43, wherein the composition is introduced to the subject intravenously, intramuscularly, intrathecally, intrarterially, intradermally, subcutaneously, intra-pleurally, intra-cranially, intra-ocularly or mucosally.

45. The method of any of claims 1-44, wherein the fibroblast regenerative cells are allogeneic with respect to the subject.

46. The method of any of claims 1-44, wherein the fibroblast regenerative cell or population thereof are autologous with respect to the subject.

47. The method of any of claims 1-46, wherein the fibroblast regenerative cells are plastic adherent.

48. The method of any of claims 1-46, wherein the fibroblast regenerative cell, population or progeny thereof is cryopreserved prior to the administering step.

49. The method of any of claims 1-48, wherein the subject is an animal.

50. The method of any of claims 1-49, wherein the composition further comprises stem cells.

51. The method of any of claims 1-50, wherein the population comprises at least 1×10², 1×10⁶, 1×10⁹, 1×10¹⁰, 1×10¹², 1×10¹⁴ stem cells or any amount in between.

52. The method of claim 50 or 51, wherein the stem cells are mesenchymal cells, embryonic stem cells or differentiated cells.

53. The method of any one of claims 50-52, wherein the stem cells are myocytes, adipocytes, ectodermal cells, muscle cells, osteoblasts, chondrocytes, endothelial cells, fibroblasts, pancreatic cells, hepatocytes, bile duct cells, bone marrow cells, neural cells, or genitourinary cells.

54. The method of any of claims 1-53, wherein the fibroblast regenerative cell does not express at least one or more of MHC class I, MHC class II, CD45, CD13, CD49c, CD66b, CD73, CD105 or CD90 cell surface proteins.

55. An in vitro method of producing a fibroblast regenerative cell or a population thereof, the method comprises selecting and/or expanding a fibroblast cell that expresses CD117 and/or CD105, wherein the fibroblast cell has increased regenerative activity as compared to a control fibroblast cell.

56. The in vitro method of claim 55, wherein the method further comprises a step of selecting the fibroblast regenerative cell for CD34 expression.

57. The in vitro method of claim 55, wherein the method further comprises a step of selecting the fibroblast regenerative cell for rhodamine 123 efflux activity.

58. The in vitro method of claim 55, wherein the method further comprises a step of selecting the fibroblast regenerative cell for at least one additional marker selected from the group consisting of Oct-4, CD-34, KLF-4, Nanog, Sox-2, Rex-1, GDF-3, and Stella.

59. The in vitro method of any of claims 55-58, wherein the method further comprises a step of selecting the fibroblast regenerative cell for enhanced expression of GDF-11 as compared to a control fibroblast cell.

60. The in vitro method of any of claims 55-59, wherein the fibroblast regenerative does not express at least one or more of MHC class I, MHC class II, CD45, CD13, CD49c, CD66b, CD73, CD105 or CD90 cell surface proteins.

61. The in vitro method of any of claims 55-60, wherein the fibroblast cell is derived from a tissue having regenerative properties.

62. The in vitro method of any of claims 55-61, wherein the fibroblast cell is derived from placental tissue, umbilical cord tissue, endometrial cells, Wharton's jelly, bone marrow or adipose tissue.

63. The in vitro method of any of claims 55-62, wherein the population comprises 0.01% to 5% freshly extracted fibroblasts.

64. The in vitro method of any of claims 55-63, wherein the method further comprises the step of preparing a therapeutically effective amount of the fibroblast regenerative cell and providing the therapeutically effective amount of the fibroblast regenerative cell, population thereof, progeny thereof, or conditioned medium thereof to a subject in need thereof.

65. An in vitro method of forming a neural cell, comprising the steps of:
obtaining a fibroblast regenerative cell or a population thereof by selecting a fibroblast cell that expresses CD117 and/or CD105, wherein the fibroblast cell has increased regenerative activity as compared to a control fibroblast cell; and
culturing said fibroblast regenerative cell with a combination of at least two of bFGF, FGF-8, SHH, or BDNF.

66. An in vitro method of forming a hepatocyte cell, comprising the steps of:
obtaining a fibroblast regenerative cell or a population thereof by selecting and expanding a fibroblast cell that expresses CD117 and/or CD105, wherein the fibroblast cell has increased regenerative activity as compared to a control fibroblast cell; and
culturing said fibroblast regenerative cell with hepatocyte growth factor (HGF) and/or FGF-4.

67. An in vitro method of forming an endothelial cell, comprising the steps of:
obtaining a fibroblast regenerative cell or a population thereof by selecting a fibroblast cell that expresses CD117 and/or CD105, wherein the fibroblast cell has increased regenerative activity as compared to a control fibroblast cell; and
culturing said fibroblast regenerative cell with vascular endothelial growth factor (VEGF).

68. An in vitro method of forming a hematopoietic cell, comprising the steps of:
obtaining a fibroblast regenerative cell or a population thereof by selecting a fibroblast cell that expresses CD117 and/or CD105, wherein the fibroblast cell has increased regenerative activity as compared to a control fibroblast cell; and
culturing said fibroblast regenerative cell with a combination of at least two of bone morphogenic protein-4 (BMP4), VEGF, bFGF, stem cell factor (SCF), Flt3L, hyper IL6, thrombopoietin (TPO) or erythropoietin (EPO).

69. The in vitro method of any of claims 65-68, wherein the method further comprises a step of selecting the fibroblast regenerative cell for CD34 expression.

70. The in vitro method of any of claims 65-69, wherein the method further comprises a step of selecting the fibroblast regenerative cell for rhodamine 123 efflux activity.

71. The in vitro method of any of claims 65-70, wherein the method further comprises a step of selecting the fibroblast regenerative cell for at least one additional marker selected from the group consisting of Oct-4, CD-34, KLF-4, Nanog, Sox-2, Rex-1, GDF-3, IL-10, Stella, and a combination thereof.

72. The in vitro method of any of claims 65-71, wherein the method further comprises a step of selecting the fibroblast regenerative cell for enhanced expression of GDF-11 as compared to a control fibroblast cell.

73. The in vitro method of any of claims 65-72, wherein the fibroblast regenerative does not express at least one or more of MHC class I, MHC class II, CD45, CD13, CD49c, CD66b, CD73, CD105 or CD90 cell surface proteins.

74. A composition comprising an isolated fibroblast cell, a population thereof, a progeny thereof or a conditioned medium thereof wherein the fibroblast cell has an increased regenerative activity compared to a control fibroblast cell.

75. The composition of claim 74, wherein the fibroblast cell expresses CD105 marker and/or CD117 marker.

76. The composition of claim 74 or 75, wherein the fibroblast cell further comprises a rhodamine 123 efflux activity.

77. The composition of any of claims 74-76, wherein the fibroblast cell expresses at least one additional marker selected from the group consisting of Oct-4, CD-34, KLF-4, Nanog, Sox-2, Rex-1, GDF-3, and Stella.

78. The composition of any of claims 74-77, wherein the fibroblast cell has enhanced expression of GDF-11 as compared to a control cell.

79. The composition of any of claims 74-78, wherein the fibroblast cell is derived from a tissue having regenerative properties.

80. The composition of any of claims 74-79, wherein the fibroblast cell is derived from placental tissue, umbilical cord tissue, endometrial cells, Wharton's jelly, bone marrow or adipose tissue.

81. The composition of any of claims 74-80, wherein the fibroblast regenerative cell, population or progeny thereof , or conditioned medium thereof is cryopreserved.

82. The composition of any of claims 74-81, wherein the population comprises at least 1×10², 1×10⁶, 1×10⁹, 1×10¹⁰, 1×10¹², 1×10¹⁴ stem cells or any amount in between.

83. The composition of any of claims 74-82, wherein the composition further comprises a bioactive compound.

84. The composition of claim 83, wherein the bioactive compound is a growth factor, a cytokine, an antibody, an antibody fragment, an organic molecule of a mass of less than 5000 daltons.

85. The composition of any of claims 74-84, wherein the composition is in a NaCl solution.

86. The composition of claim 74-84, wherein the composition is in a 0.8%-1% NaCl solution.

87. The composition of any of claims 74-86, wherein the composition further includes exogenous mitochondria.

88. The composition of claim 87, wherein the exogenous mitochondria are isolated and substantially pure from other cellular components.

89. The composition of any of claims 87-88, wherein the mitochondria are administered to the fibroblast cell by lipid fusion, polyethylene glycol-mediated fusion or by electroporation.

90. The composition of any of claims 87-89, wherein the mitochondria are encapsulated or treated with agents to facilitate the incorporation of the mitochondria into the fibroblast cell.

100. The composition of any of claims 87-90, wherein the composition further includes vitamin A, vitamin C, vitamin D, vitamin E, vitamin K, folic acid, choline, vitamin B1, vitamin B2, vitamin B5, vitamin B6, vitamin B12, biotin, nicotinamide, betacarotene, coenzyme Q, selenium, superoxide dismutase, glutathione peroxide, uridine, creatine succinate, pyruvate, dihydroxyacetone), acetyl-L-carnitine, alpha-lipoic acid, cardiolipin, omega fatty acid, lithium carbonate, lithium citrate, calcium, or a combination thereof.

101. The composition of any of claims 87-91, wherein the mitochondria is syngeneic mitochondria, allogeneic mitochondria, or xenogeneic mitochondria.

102. The composition of any of claims 87-101, wherein the mitochondria are derived from stem cells, or from cells less differentiated as compared to the fibroblast cell.

103. The composition of any of claims 74-102, wherein the regenerative activity is the ability to stimulate angiogenesis.

104. The composition of claim 103, wherein angiogenesis further comprises stimulation of human umbilical vein endothelial cell (HUVEC) proliferation.

105. The composition of claim 103 or 104, wherein said angiogenesis comprises the production of collateral blood vessels.

106. The composition of claim 105, wherein said collateral blood vessels are in an ischemic cardiac tissue.

107. The composition of claim 105, wherein said collateral blood vessels are in an ischemic limb tissue.

108. The composition of claim 105, wherein said collateral blood vessels are surrounding an occluded blood vessel.

109. A kit comprising the composition of any of claims 74-108.

110. A pharmaceutical formulation comprising the composition of any of claims 74-109.

111. An in vitro isolated fibroblast regenerative cell or population thereof capable of proliferating and differentiating into ectoderm, mesoderm, or endoderm, wherein the isolated fibroblast regenerative cell expresses at least one of Oct-4, Nanog, Sox-2, KLF4, c-Myc, Rex-1, GDF-3, LIF receptor, CD105, CD117, CD344, IL-10, or Stella markers, and does not express at least one of MHC class I, MHC class II, CD45, CD13, CD49c, CD66b, CD73, CD105, or CD90 cell surface proteins.

112. A master cell bank comprising a plurality of packaged population of regenerative fibroblast cells, capable of proliferating and differentiating into ectoderm, mesoderm, or endoderm, wherein the isolated fibroblast regenerative cell expresses at least one of Oct-4, Nanog, Sox-2, KLF4, c-Myc, Rex-1, GDF-3, LIF receptor, CD105, CD117, CD344, IL-10, and Stella, and does not express at least one of MHC class I, MHC class II, CD45, CD13, CD49c, CD66b, CD73, CD105, or CD90 cell surface proteins.

113. The master cell bank of claim 112, wherein each packaged population includes at least 1×10² or more of the cells of previous aspect.

114. A method for isolating a population of regenerative fibroblast cells, the method comprising:
providing a tissue with regenerative activity; and
enriching for a population of cells that are about 6-12 micrometers in size, wherein the fibroblast regenerative cells express at least one of Oct-4, Nanog, Sox-2, KLF4, c-Myc, Rex-1, GDF-3, LIF receptor, CD105, CD117, CD344, IL-10, and Stella, and does not express at least one of MHC class I, MHC class II, CD45, CD13, CD49c, CD66b, CD73, CD105, or CD90 cell surface proteins.

115. The method of claim 114, wherein the method optionally includes the step of depleting cells from the population expressing stem cell surface markers or MHC proteins, thereby isolating a population of stem cells.

116. The method of claim 115, wherein the cells to be depleted express MHC class I, CD66b, glycophorin a, or glycophorin b.

117. The method of claim 114, wherein the method optionally includes cryopreserving the cells.

118. The method of claim 114, wherein the method optionally includes transfecting the cells with a polynucleotide vector containing a stem cell-specific promoter operably linked to a reporter or selection gene.

119. The method of claim 118, wherein the cell-specific promoter is an Oct-4, Nanog, Sox-9, GDF3, Rex-1, or Sox-2 promoter.

120. The method of any one of claims 114-119, wherein the method further includes the step of enriching the population for the regenerative fibroblast cells using expression of a reporter or selection gene.

121. The method of any one of claims 114-120, wherein the method further includes the step of enriching the population of the regenerative fibroblast cells by flow cytometry.

122. The method of any one of claims 114-121, wherein the method further includes the steps of:
contacting the cells with a detectable compound which enters said cells, the compound being selectively detectable in proliferating and non-proliferating cells; and
enriching the population of cells for the proliferating cells.

123. The method of claim 122, wherein the detectable compound is carboxyfluorescein diacetate, succinimidyl ester, or Aldefluor.

124. The method of any of claims 114-123, wherein the method further includes culturing the cells under conditions which form tissue aggregate bodies.

125. The method of any of claims 114-124, wherein the further includes separating cell types such as granulocytes, T-cells, B-cells, NK-cell, red blood cells, or any combination thereof, from the fibroblast regenerative cells.

126. The method of claim 125, wherein the separating the cell types is done by cell depletion.

127. The method of any of claims 114-126, wherein the method further includes culturing the population of fibroblast regenerative cells under conditions which support proliferation of the cells.

128. The method of any of claims 114-127, wherein the cells are cryopreserved.

129. A cell produced by the method of any of claims 114-128.

130. A method of identifying a fibroblast regenerative cell, the method comprising the steps of:
introducing into a cell a vector comprising a fibroblast cell-specific promoter coupled to at least one selectable marker gene;
expressing the selectable marker gene from the cell specific promoter in the cell; and
detecting expression of the marker gene in the cell, thereby identifying the fibroblast regenerative cell, wherein said fibroblast regenerative cell does not express at least one or more of MHC class I, MHC class II, CD44, CD45, CD13, CD34, CD49c, CD66b, CD73, CD105, and CD90 cell surface proteins; and said fibroblast regenerative cell expresses at least one or more of Oct-4, Nanog, Sox-2, Rex-1, GDF-3, Stella, FoxD3, or Polycomb embryonic transcription factors, and wherein said fibroblast regenerative cell is capable of differentiating into mesoderm, ectoderm, and/or endoderm.

131. The method of claim 130, wherein the method further comprises the step of isolating the fibroblast regenerative cell.

132. The method of claim 130 or 131, wherein the fibroblast regenerative cell is derived from the bodily fluid or from the tissue of a mammal.

133. The method of claim 132, wherein the bodily fluid is synovial fluid or blood.

134. The method of any one of claims 130-132, wherein the mammal is a human.

135. The method of any of claims 130-134, wherein the fibroblast cell does not express CD13, CD44, CD90 or a combination thereof.

136. The method of any of claims 130-135, wherein the fibroblast cell-specific promoter is an Oct-4 promoter, a Nanog promoter, a Sox-2 promoter, a Rex-1 promoter, a GDF-3 promoter, aStella promoter, a FoxD3 promoter, a Polycomb Repressor Complex 2 promoter, or aCTCF promoter.

137. The method of any of claims 130-136, wherein the fibroblast cell-specific promoter is flanked by loxP sites.

138. The method of any of claims 130-137, wherein the vector is a retroviral vector.

139. The method of any of claims 130-138, wherein the selectable marker gene encodes a fluorescent protein.

140. The method of claim 139, wherein the fluorescent protein is Green Fluorescent Protein (GFP).

141. The method of any of claims 130-140, further including the step of transfecting the regenerative fibroblast cells with OCT-4 transcription factor thereby enhancing the regenerative activity of the fibroblast cells.

142. The method of any of claims 130-140, further including the step of fusing the regenerative fibroblast cells with cells having a pluripotent ability thereby generating fibroblasts with enhanced regenerative activity.

143. The method of any one of claims 130-142, wherein the method further comprises the steps of: selecting fibroblast cells expressing CD105 and/or CD 117; and
transfecting the fibroblast cells expressing CD105 and/or CD 117 with permeant NANOG gene.

144. The method of any of claims 130-144, wherein the vector two selectable marker genes.

145. The method of claim 144, wherein the two selectable marker genes are a fluorescent protein and a protein sensitive to drug selection.

146. The method of claim 144, wherein one of the selective marker genes encodes a cell surface protein.

147. The method of any one of claims 130-146, wherein the fibroblast regenerative cell further comprises a rhodamine 123 efflux activity.

148. The method of any one of claims 130-147, wherein the fibroblast regenerative cell has enhanced expression of GDF-11 as compared to a control cell.

149. The method of any one of claims 130-148, wherein the fibroblast regenerative cell is derived from a tissue having regenerative properties.

150. The method of any one of claims 130-150, wherein the fibroblast regenerative cell is derived from placental tissue, umbilical cord tissue, endometrial cells, Wharton's jelly, bone marrow or adipose tissue.

151. A method of generating a regenerative fibroblast cell comprising the steps of introducing into a population of fibroblasts a vector comprising a regenerative cell-specific promoter coupled to at least one selectable marker gene, wherein said regenerative cell does not express MHC class I, MHC class II, CD44, CD45, CD13, CD34, CD49c, CD73, CD105 and CD90 cell surface proteins;
expressing the selectable marker gene from the regenerative-cell specific promoter in said fibroblast population; and
detecting expression of the marker gene in the regenerative fibroblast cell.

152. The method of claim 151, wherein the selectable marker gene encodes a fluorescent protein, a protein sensitive to drug selection or a cell surface protein.

153. The method of claim 151 or 152, wherein the method further includes the step of transfecting the regenerative fibroblast cell with a nucleic acid encoding OCT-4 transcription factor thereby enhancing the regenerative activity of the fibroblast cell.

154. The method of any one of claims 151-153, further including the step of fusing the regenerative fibroblast cells with cells having a pluripotent ability thereby generating fibroblasts with enhanced regenerative activity.

155. The method of claim 151, wherein the method further comprises the steps of:
selecting fibroblast cells expressing CD105 and/or CD 117; and
transfecting the fibroblast cells with permeant NANOG gene.

156. A fibroblast regenerative cell isolated by the method of any one of claims 151-155.
